(19) United States
(12) Patent Application Publication
(10) Pub. No.: US 2024/0301461 A1
(43) Pub. Date: Sep. 12, 2024

(54) Recombinant Escherichia Coli for Expressing Synthesis Pathway of Asiaticoside and Application Thereof

(71) Applicant: Jiangnan University, Wuxi (CN)

(72) Inventors: Jingwen Zhou, Wuxi (CN); Weizhu Zeng, Wuxi (CN); Xingying Zhao, Wuxi (CN); Sha Xu, Wuxi (CN); Jian Chen, Wuxi (CN)

(21) Appl. No.: 18/414,523

(22) Filed: Jan. 17, 2024

(30) Foreign Application Priority Data
Jan. 17, 2023 (CN) 2023100674925
Jan. 17, 2023 (CN) 2023100818103

(51) Int. Cl. C12P 19/48; C12N 1/20; C12N 9/10; C12R 1/19

(52) U.S. Cl. CPC C12P 19/485; C12N 1/205; C12N 9/1051; C12N 2527/00; C12R 2001/19

(57) ABSTRACT

The present disclosure discloses recombinant Escherichia coli for expressing a synthesis pathway of asiaticoside and application thereof, and belongs to the field of bioengineering. Rhamnosyltransferase with an amino acid sequence shown in any one of SEQ ID NO: 25 to SEQ ID NO: 29, glucosyltransferase with an amino acid sequence shown in any one of SEQ ID NO: 10 to SEQ ID NO: 17 and UGT73AH1 reported in a document are transferred into E. coli BL21 (DE3)Δpgi to realize co-expression. Fermentation results show that all 5 rhamnosyltransferases screened in the present disclosure can achieve effects, and a unique new peak appears at 0.596 min, which is consistent with a characteristic ion flow of an asiaticoside standard product. According to the present disclosure, barriers of the prior art are broken through, a new biosynthesis method for asiaticoside is provided, and industrialization of biosynthesis of asiaticoside becomes possible.

### REFERENCE TO SEQUENCE LISTING

The instant application contains a Sequence Listing in XML format as a file named “YGHY-2023-51-SEQ.xml”, created on Jan. 12, 2024, of 90.4 kB in size, and which is hereby incorporated by reference in its entirety.

### TECHNICAL FIELD

The present disclosure relates to recombinant Escherichia Coli for expressing a synthesis pathway of asiaticoside and application thereof, and belongs to the field of bioengineering.

### BACKGROUND

As a dry whole herb of umbelliferae plants, Centella asiatica includes the chemical components of triterpene saponins, triterpenic acids, polyalkynenes, volatile oils and the like. Moreover, the Centella asiatica is rich in a variety of bioactive substances, including the main components of triterpenoids and saponin compounds thereof, asiaticoside B, madecassoside and asiaticoside.

Triterpenoid compounds have been widely found in fungi, ferns, monocotyledons, dicotyledons and animals, especially in dicotyledons. The main bioactive substances of leaf extracts of the Centella asiatica are the saponins. The asiaticoside has a wide range of biological activities, such as resistance to cancer, resistance to inflammation, promotion of wound healing, resistance to diabetes mellitus, resistance to oxidation, liver protection, resistance to hepatitis C virus (HCV) and neuroprotection. However, as an important secondary metabolite of plants, the asiaticoside has a complex structure and a relatively low content in plants. In addition, most of these compounds can be mixed with compounds with similar structures. Therefore, it is difficult to obtain a large number of triterpenoid monomers by plant extraction or chemical synthesis, thereby limiting application of the compounds in food, medicine, cosmetic products and other fields. With rapid development of sequencing technologies, more and more enzymes in synthesis pathways of terpenoids have been excavated and identified, and the synthesis pathways of terpenoids have been resolved. In addition, with rapid development of synthetic biology, construction of microbial cell factories to produce the terpenoids and other compounds with great medical values has become a research hotspot.

In the past few decades, relatively little progress has been made on functional characterization of a gene UGT involved in biosynthesis of the triterpenoid compounds, which may be related to plant genomes capable of encoding a large number of UGT homologues. At present, synthesis pathways of the asiaticoside have not been fully resolved, and it is believed through research that the asiaticoside is prepared from asiatic acid by glycosylation in three steps. It is reported by Costa et al. that UGT73AD1 has the effect of connecting a carboxyl group of glucose in Centella asiatica, and it is determined through in vitro expression that the UGT73AD1 can be used for specifically glycosylating asiatic acid and madecassic acid. According to Kim et al., glycosyltransferase UGT73AH1 is identified from Centella asiatica (L.) Urban, and the enzyme can be used for glycosylating C28-COOH of asiatic acid to produce corresponding monoglycoside. However, whether the enzyme has a catalytic effect on C-2α, C-3β and C23-OH at other sites is unclear. In addition, glycosyltransferase in last two steps of the three steps for glycosylation is still unknown.

### SUMMARY

In the present disclosure, recombinant E. coli capable of synthesizing asiaticoside is constructed by subjecting a Centella asiatica μlant to second generation transcriptome sequencing, excavating glucosyltransferase and rhamnosyltransferase with potential functions by BLAST comparison, protein modeling, molecular docking, phylogenetic analysis and other bioinformatics means, performing expression verification in E. coli and then adding asiatic acid as an exogenous substrate.

The first objective of the present disclosure is to provide application of glucosyltransferase in preparation of asiaticodiglycoside or asiaticoside. The glucosyltransferase is shown in the following (a) or (b):
(a) having an amino acid sequence shown in any one of SEQ ID NO: 10 to SEQ ID NO: 17; and
(b) being a protein derived from (a) that is prepared by subjecting the amino acid sequence in (a) to substitution, deletion or addition of one or more of amino acids and has activities of glucosyltransferase.

In one embodiment of the present disclosure, a gene for encoding the glucosyltransferase has a nucleotide sequence shown in any one of SEQ ID NO: 1 to SEQ ID NO: 8.

The second objective of the present disclosure is to provide recombinant E. coli. E. coli is used as a host for overexpressing glucosyltransferase and glycosyltransferase UGT73AH1.

In one embodiment of the present disclosure, the glucosyltransferase is shown in the following (a) or (b):
(a) having an amino acid sequence shown in any one of SEQ ID NO: 10 to SEQ ID NO: 17; and
(b) being a protein derived from (a) that is prepared by subjecting the amino acid sequence in (a) to substitution, deletion or addition of one or more of amino acids and has activities of glucosyltransferase.

In one embodiment of the present disclosure, the recombinant E. coli is obtained by knocking out a gene pgi for encoding a glucose phosphate isomerase protein on a genome.

In one embodiment of the present disclosure, the gene pgi for encoding a glucose phosphate isomerase protein has a nucleotide sequence shown in SEQ ID NO: 18.

In one embodiment of the present disclosure, the glycosyltransferase UGT73AH1 is derived from Centella asiatica (L.) Urban.

In one embodiment of the present disclosure, the glycosyltransferase UGT73AH1 has a nucleotide sequence shown in SEQ ID NO: 9 and an amino acid sequence shown in SEQ ID NO: 19.

In one embodiment of the present disclosure, E. coli BL21 (DE3) is used as a host for the recombinant E. coli.

In one embodiment of the present disclosure, the recombinant E. coli is used for overexpressing rhamnosyltransferase with an amino acid sequence shown in any one of SEQ ID NO: 25 to SEQ ID NO: 29.

In one embodiment of the present disclosure, the recombinant E. coli is used for expressing rhamnosyltransferase with pRSFDuet-1 as a vector and co-expressing glucosyltransferase and glycosyltransferase UGT73AH1 with pETDuet-1.

In one embodiment of the present disclosure, E. coli BL21 (DE3) is used as a host for the recombinant E. coli.

The third objective of the present disclosure is to provide application of the recombinant E. coli in synthesis of asiaticodiglycoside or asiaticoside.

The fourth objective of the present disclosure is to provide a method for producing asiaticodiglycoside. The method includes inoculating a seed liquid of the recombinant E. coli into a fermentation culture medium, performing shaking culture at 200-240 rpm at 35-38° C. until an OD₆₀₀ value is 0.6-0.8, adding isopropyl thiogalactoside to a final concentration of 0.4-0.6 μmol/L, adding asiatic acid with a final concentration of 150-250 mg/L after 4-8 h, and then performing shaking culture continuously at 200-240 rpm at 28-32° C. for 40-60 h;

or with glucosyltransferase and glycosyltransferase UGT73AH1 as a catalyst, carrying out a reaction in a reaction solution containing uridine diphosphate glucose (UDPG) as a glycosyl donor with a final concentration of 0.8-1.2 g/L and asiatic acid as a substrate with a concentration of 150-250 mg/L at 35-38° C. for 4-10 h.

In one embodiment of the present disclosure, the fermentation culture medium includes 15-25 g/L of glucose, 4-6 g/L of glycerol, 14-18 g/L of K₂HPO₄·3H₂O, 1-3 g/L of KH₂PO₄, 20-30 g/L of yeast powder and 10-15 g/L of peptone.

In one embodiment of the present disclosure, the glucosyltransferase is shown in the following (a) or (b):
(a) having an amino acid sequence shown in any one of SEQ ID NO: 10 to SEQ ID NO: 17; and
(b) being a protein derived from (a) that is prepared by subjecting the amino acid sequence in (a) to substitution, deletion or addition of one or more of amino acids and has activities of glucosyltransferase.

In one embodiment of the present disclosure, the glycosyltransferase UGT73AH1 is derived from Centella asiatica (L.) Urban.

In one embodiment of the present disclosure, the glycosyltransferase UGT73AH1 has an amino acid sequence shown in SEQ ID NO: 19.

The fifth objective of the present disclosure is to provide application of rhamnosyltransferase in synthesis of asiaticoside or a product containing asiaticosid.

In one embodiment of the present disclosure, the rhamnosyltransferase is shown in the following (a) or (b):
(a) having an amino acid sequence shown in any one of SEQ ID NO: 25 to SEQ ID NO: 29; and
(b) being a protein derived from (a) that is prepared by subjecting the amino acid sequence in (a) to substitution, deletion or addition of one or more of amino acids and has activities of glucosyltransferase.

In one embodiment of the present disclosure, a gene for encoding the rhamnosyltransferase has a nucleotide sequence shown in any one of SEQ ID NO: 20 to SEQ ID NO: 24.

The present disclosure has the following beneficial effects.

(1) According to the present disclosure, genes for encoding glucosyltransferase that are screened by transcriptome sequencing of Centella asiatica are linked to an E. coli expression vector pETDuet-1, respectively, followed by induced expression with an E. coli host BL21 (DE3)Δpgi. Induction is performed with 0.5 μmol/L of isopropyl thiogalactoside (IPTG) at 16° C. for 20 h, and cells are collected and subjected to ultrasonic crushing. SDS-PAGE results show that all 8 glucosyltransferases can be expressed normally. Genes for encoding rhamnosyltransferase that are screened from a transcriptome of Centella asiatica are linked to an E. coli expression vector pRSFDuet-1, respectively, followed by induced expression with an E. coli host BL21 (DE3)Δpgi. Induction is performed with 0.5 μmol/L of IPTG at 20° C. for 20 h, and cells are collected and subjected to ultrasonic crushing. SDS-PAGE results show that all 5 rhamnosyltransferases can be expressed normally.

(2) The screened genes for encoding the 8 glucosyltransferases are linked to an E. coli expression vector pETDuet-1, respectively, meanwhile, glucosyltransferase UGT73AH1 with a C-28 site reported in a document is linked to an E. coli expression vector pET28a, and recombinant E. coli is constructed with E. coli BL21 (DE3)Δpgi as a host. Induced expression is performed under certain culture conditions, and shake flask fermentation is performed with an empty vector pETDuet-1 as a blank control. 20 g/L of glucose and asiatic acid as a substrate with a final concentration of 200 mg/L are added into a TB culture medium, and samples are collected and detected at 24 h. As can be seen from mass spectrograms, when UGT73AH1, UGT73C7 and UGT73C8 are transferred into the strain BL21 (DE3)Δpgi at the same time, a new peak different from that of the blank control appears at 7.92 min, and mass spectrum data same as that of asiaticodiglycoside are obtained, determining that asiaticodiglycoside is produced in a fermentation solution.

(3) The screened genes for encoding the 8 glucosyltransferases are linked to an E. coli expression vector pETDuet-1, respectively, and recombinant E. coli is constructed with E. coli BL21 (DE3)Δpgi as a host. Induced expression is performed under certain culture conditions with an empty vector pETDuet-1 as a blank control, induction is performed with 0.5 μmol/L of IPTG at 16° C. for 20 h, and cells are collected and subjected to ultrasonic crushing to obtain a crude enzyme solution. The volume of the total enzyme reaction system is 500 μl, the volume of the crude enzyme solution is 100 μl, the final concentration of the added UDPG is 1 g/L, the final concentration of asiatic acid added as a substrate is 200 mg/L, and the system is supplemented with a PBS buffer solution to 500 μl to carry out a reaction at 37° C. for 6 h. As can be seen from mass spectrograms, when UGT73AH1, UGT73C7 and UGT73C8 are transferred into the strain BL21 (DE3)Δpgi at the same time, a new peak different from that of the blank control appears at 7.92 min, and mass spectrum data same as that of asiaticodiglycoside are obtained, determining that asiaticodiglycoside is produced in a reaction solution.

(4) According to the present disclosure, the genes for encoding the 5 rhamnosyltransferases that are screened from a transcriptome of Centella asiatica and rhamnose isomerase VvRHM derived from Vitis vinifera are linked to an E. coli expression vector pRSFDuet-1, respectively, and glucosyltransferase UGT73AH1 with a C-28 site reported in a document and glucosyltransferase UGT73 (UGT73C7 or UGT73C8) are linked to an E. coli expression vector pETDuet-1 and transferred into an E. coli host BL21 (DE3)Δpgi to construct recombinant E. coli. The recombinant E. coli is inoculated into a TB culture medium containing 20 g/L of glucose, where the final concentration of asiatic acid added as a substrate is 200 mg/L. After induced expression, samples are collected and detected at 24 h. As can be seen from mass spectrograms, all the 5 rhamnosyltransferases screened from the transcriptome can achieve effects, and a unique new peak appears at 0.596 min, which is consistent with a characteristic ion flow of an asiaticoside standard product. According to the present disclosure, barriers of the prior art are broken through, a new biosynthesis method for asiaticoside is provided, and industrialization of biosynthesis of asiaticoside becomes possible.

### BRIEF DESCRIPTION OF FIGURES

FIG. 1 shows SDS-PAGE electrophoresis results of recombinant E. coli UGT73 in expression, where FIG. A shows a supernatant, and FIG. B shows a precipitate.

FIG. 2A shows a liquid phase of recombinant E. coli UGT73 and UGT73AH1 in shake flask fermentation; FIG. 2B shows a liquid phase of a UGTs glycosylation product fermented by recombinant E. coli UGT73AH1 in shake flask fermentation.

FIG. 3A shows mass spectrograms on positive ion fragments of recombinant E. coli UGT73 and UGT73AH1 in shake flask fermentation; FIG. 3B shows mass spectrograms on negative ion fragments of recombinant E. coli UGT73 and UGT73AH1 in shake flask fermentation.

FIG. 4A shows mass spectrograms on positive ion fragments of crude enzyme solutions of recombinant E. coli UGT73 and UGT73AH1 in a reaction; FIG. 4B shows mass spectrograms on negative ion fragments of crude enzyme solutions of recombinant E. coli UGT73 and UGT73AH1 in a reaction.

FIG. 5 shows SDS-PAGE electrophoresis results of recombinant E. coli RRT in expression.

FIG. 6A-D shows liquid phase results of recombinant E. coli RRT and VvRHM in shake flask fermentation.

FIG. 7 shows mass spectrometry results of recombinant E. coli RRT and VvRHM in shake flask fermentation.

### DETAILED DESCRIPTION

### (One) Culture Media

A seed culture medium (LB) includes: 10 g/L of peptone, 5 g/L of a yeast extract and 5 g/L of sodium chloride; and 2% (mass fraction) of agar powder was added into a solid culture medium.

A fermentation culture medium (TB) includes: 20 g/L of glucose, 5 g/L glycerol, 16.4 g/L of K₂HPO₄·3H₂O, 2.31 g/L of KH₂PO₄, 24 g/L of yeast powder and 12 g/L of peptone. 20 g/L of the glucose was sterilized separately and mixed uniformly before inoculation.

### (Two) PCR Reaction System and Amplification Conditions

1 μL (10 μM) of a forward primer, 1 μL (10 μM) of a reverse primer, 20 ng of template DNA and 25 μL of 2×Phanta Max Master Mix were used, and double distilled water was added to 50 μL. Amplification conditions were as follows: pre-denaturation at 95° C. for 3 min, followed by 30 cycles (at 95° C. for 15 s, at 55° C. for 15 s and at 72° C. for 15 s) and continuous extension at 72° C. for 10 min.

(Three) Preparation of E. coli Competent Cells

E. coli BL21 (DE3) was subjected to gene editing by using a CRISPR/Cas9 system to construct E. coli BL21 (DE3)Δpgi. A plasmid pTarget used for knockout was constructed by a PCR mediated point mutation technology, and a template plasmid pTarget were stored in a laboratory. Primers P21pgi-F and P21pgi-R were used for constructing a plasmid pTarget-pgi, and PPGI-UPARM-F/R and PPGI-downARM-F/R were used for constructing homologous arms used for knockout. Specific operation methods are referred to the document: Li Q, Sun B, Chen J, et al. A modified pCas/pTargetF system for CRISPR-Cas9-assisted genome editing in Escherichia coli [J]. Acta Biochimica et Biophysica Sinica: English version, 2021, 53(5):8.

P21pgi-F:
(SEQ ID NO: 64)
AGTTGCTGGCGCTGATTGGCATCGTTTTAGAGCTAGAAATAGCAAGTTA
AAATAAGGCT;
P21pgi-R:
(SEQ ID NO: 65)
AAACGATGCCAATCAGCGCCAGCACTAGTATTATACCTAGGACTGAGCT
AG;
21-PGI-UPARM-F:
(SEQ ID NO: 66)
cctcgtgtcaggggatccattttc;
21-PGI-UPARM-R:
(SEQ ID NO: 67)
tgatccggcaaacaaaccaccgctggtagccacggcgcggttttcagtg
c;
21-PGI-DOWNARM-F:
(SEQ ID NO: 68)
gctaccagcggtggtttgtttgccggatcattgagcaggaatatcgtga
tcagg;
21-PGIDOWNARM-R:
(SEQ ID NO: 69)
tttacccaaaaacatttcgggcg;

BL21 (DE3)Δpgi in a glycerol tube was streaked on a corresponding LB plate and cultured overnight at 37° C. (for about 12 h). After 12 h, flat, round and vigorously growing bacteria were selected, inoculated into a 50 mL shake flask containing 5 mL of an LB culture medium and cultured at 220 rpm at 37° C. for about 8-10 h. The bacteria were inoculated into a 250 mL conical flask containing 50 mL of LB at an inoculation amount of 1% and cultured at 220 rpm at 37° C. for about 2 h until an OD₆₀₀ value was 0.6-0.8. A bacterial solution was transferred to a 50 mL centrifuge tube, placed on ice for about 10-15 min and centrifuged at 4,000 rpm at 4° C. for 5 min to remove a supernatant. 5 mL of a solution A was added for resuspension, and centrifugation was performed at 4,000 rpm at 4° C. for 5 min to remove a supernatant. Then, 5 mL of a solution B was added to resuspend the bacteria, and a resulting product was packaged in 100 μL/part and store at −80° C.

(Four) Transformation of E. coli

E. coli competent cells were thawed on ice. 10 μL of a recombinant product was added into 100 μL of the competent cells and subjected to standing on ice for 30 min. A resulting mixture was subjected to heat shock in a water bath pot at 42° C. for 45 s, followed by standing on ice for 2 min. 1 mL of an LB culture medium was added, and incubation was performed at 220 rpm at 37° C. for 60 min. Then, centrifugation was performed at 4,000 rpm for 2 min to remove 900 μL of a supernatant. The bacteria were resuspended with the remaining culture medium and then coated on a corresponding resistant plate.

(Five) Identification of asiaticodiglycoside: After completion of fermentation, 2 mL of a fermentation solution was taken, added into an equal volume of methanol, violently shaken and uniformly mixed, followed by centrifugation at 14,000 r/min for 10 min. A supernatant was taken and filtered with a 0.22 μm organic phase filter membrane, and a product was identified by UPLC-IT-TOF/MS of Shimadzu.

(Six) Determination of asiaticodiglycoside by high performance liquid chromatography (HPLC): A Thermo Fisher C18 chromatographic column (4.6 mm×250 mm, 5 m) was used for chromatographic separation; the temperature of a column oven was set to 40° C.; the injection volume was 10 μL; mobile phases were as follows: phase A: ultrapure water (0.1% of trifluoroacetic acid was added), and phase B: acetonitrile (0.1% of trifluoroacetic acid was added); the total flow rate was 1 mL/min; isocratic elution was used as an elution method, and the ratio of the phase A to the phase B was 70:30; and the wavelength of a detector was 210 nm.

(Seven) Extraction of asiaticoside: After completion of fermentation, 2 ml of a fermentation solution was taken, added into an equal volume of methanol, violently shaken and uniformly mixed, followed by centrifugation at 14,000 r/min for 10 min. A supernatant was taken and filtered with a 0.22 μm organic phase filter membrane, and a product was detected by UPLC-IT-TOF/MS of Shimadzu.

(Eight) Determination of asiaticoside by HPLC: A Thermo Fisher C18 chromatographic column (4.6 mm×250 mm, 5 m) was used for chromatographic separation; the temperature of a column oven was set to 40° C.; the injection volume was 10 μL; mobile phases were as follows: phase A: ultrapure water (0.1% of trifluoroacetic acid was added), and phase B: acetonitrile (0.1% of trifluoroacetic acid was added); the total flow rate was 1 mL/min; isocratic elution was used as an elution method, and the ratio of the phase A to the phase B was 70:30; and the wavelength of a detector was 210 nm.

### Example 1 Screening of Glucosyltransferases with Potential Functions

Through BLAST comparison between data of a transcriptome of Centella asiatica and glucosyltransferases with a C-28 site that have known functions and are derived from 17 different sources in documents, results show that 5 isoenzymes are basically homologous to 12 genes with an E value of 0.

Through comparison between sequences of the 12 genes, it can be seen that evm.model.CM025782.1.1646 [mRNA] and reported CaUGT73AH1 have differences in only one amino acid, evm.model.CM025782.1.1645 [mRNA] and evm.model.CM025782.1.1643 [mRNA] have an exactly same protein sequence, and evm.model.CM025782.1.1647 [mRNA] and CaUGT73AH1 have an exactly same protein sequence. In addition, all known glucosyltransferases with C28-COOH reported at present belong to the UGT73 family. According to KEGG analysis results is combination with BLAST comparison results, the enzymes were renumbered, as shown in Table 1 below.

TABLE 1
Glycosyltransferases with high similarity

| | | Functional annotation | Functional |
|---|---|---|---|
| Transcriptome gene ID | Number | of KEGG | annotation of GO |
| evm.model.CM025782.1.1649 | UGT73C1 | K13496 UGT73C; UDP- | GO:0008194(UDP- |
| [mRNA] | | glucosyltransferase 73C | glycosyltransferase |
| | | [EC:2.4.1.-] | activity) |
| evm.model.CM025782.1.1644 | UGT73C4 | | |
| [mRNA] | | | |
| evm.model.CM025782.1.1643 | UGT73C5 | | |
| [mRNA] | | | |
| evm.model.CM025783.1.1337 | UGT73C7 | | |
| [mRNA] | | | |
| evm.model.CM025783.1.1336 | UGT73C8 | | |
| [mRNA] | | | |
| evm.model.CM025783.1.1338 | UGT73C9 | | |
| [mRNA] | | | |
| evm.model.CM025783.1.1335 | UGT73C10 | ND | |
| [mRNA] | | | |
| evm.model.CM025781.1.1405 | UGT73C11 | ND | |
| [mRNA] | | | |

### Example 2 Gene Amplification of Glycosyltransferase and Construction of Recombinant E. coli for Expressing Glucosyltransferase

With cDNA obtained by reverse transcription of Centella asiatica as a template, priming pairs (Table 2) used for amplifying the glucosyltransferases screened in Example 1 were designed and subjected to amplification by PCR, respectively by selecting a Primer Star MasterMix high-fidelity pfu enzyme (Takara) under the following conditions: pre-denaturation at 95° C. for 3 min; at an amplification stage, 30 cycles at 95° C. for 15 s, at 60° C. for 15 s and at 72° C. for 1 min; and extension at 72° C. for 5 min. PCR products were purified to obtain target fragments UGT73C1 to UGT73C11, a vector pETDuet-1 was subjected to amplification by PCR with primers pETDuet-F and pETDuet-R, and a product was purified. The purified fragments UGT73C1 to UGT73C11 were recombined with the vector pETDuet-1 skeleton by a Gibson assembly method to obtain recombinant vectors, respectively, and the recombinant vectors were transferred into E. coli JM109. The recombinant vectors obtained were sent to Shanghai Biotech for sequencing to obtain correctly sequenced recombinant plasmids pETDuet-1-UGT73C1, pETDuet-1-UGT73C4, pETDuet-1-UGT73C5, pETDuet-1-UGT73C7, pETDuet-1-UGT73C8, pETDuet-1-UGT73C9, pETDuet-1-UGT73C10 and pETDuet-1-UGT73C11. Then, the plasmids were transferred into E. coli BL21 (DE3)Δpgi to obtain recombinant E. coli BL21 (DE3)Δpgi/pETDuet-1-UGT73C1 to BL21 (DE3)Δpgi/pETDuet-1-UGT73C11, respectively.

A primer pair (UGT73AH1-F/UGT73AH1-R, Table 2) used for amplifying UGT73AH1 derived from Centella asiatica (L.) Urban was designed. With a synthetic sequence as a template, the primer pair was subjected to amplification by PCR by selecting a Primer Star MasterMix high-fidelity pfu enzyme (Takara) under the following conditions: pre-denaturation at 95° C. for 3 min; at an amplification stage, 30 cycles at 95° C. for 15 s, at 56° C. for 15 s and at 72° C. for 1 min; and extension at 72° C. for 5 min. A PCR product was purified, a vector pET28a was subjected to amplification by PCR with 28a-F and 28a-R, and a product was purified. A purified fragment UGT73AH1 and the vector pET28a skeleton were recombined by a Gibson assembly method to obtain a recombinant vector, and the recombinant vector was transferred into E. coli JM109. The obtained vector was sequenced to obtain a correctly sequenced recombinant plasmid pET28a-UGT73AH1, and then the plasmid was transferred into E. coli BL21 (DE3)Δpgi to obtain recombinant E. coli BL21 (DE3)Δpgi/pET28a-UGT73AH1.

The recombinant plasmids pETDuet-1-UGT73C1 to pETDuet-1-UGT73C11 were transferred into the recombinant E. coli BL21 (DE3)Δpgi/pET28a-UGT73AH1, respectively to obtain a series of recombinant bacteria BL21 (DE3)Δpgi/pETDuet-1-UGT73C1/pET28a-UGT73AH1 to BL21 (DE3)Δpgi/pETDuet-1-UGT73C11/pET28a-UGT73AH1 containing UGT73AH1 and the glycosyltransferases screened in Example 1.

Primers used for constructing expression vectors of glucosyltransferases

| Primer | Sequence (5′-3′) | |
|---|---|---|
| UGT73C1-F | TAAGAAGGAGATATACCATGACCAGCAGTCAGCTGAAAG | SEQ ID NO: 30 |
| UGT73C1-R | CTGTTCGACTTAAGCATTACTTATTCAGCAGCGGACCCACA | SEQ ID NO: 31 |
| UGT73C4-F | TAAGAAGGAGATATACCATGGATGATCTCTCTTCTCTAAAACTGGGTGTTAAATT | SEQ ID NO: 32 |
| | TTTTCAAGC | |
| UGT73C4-R | CTGTTCGACTTAAGCATTACTTTCCCTTAACAAACGGCGGACTTCCAC | SEQ ID NO: 33 |
| UGT73C5-F | TTAAGAAGGAGATATACCATGGCTACCAATATTGAGCAGCAGCAGC | SEQ ID NO: 34 |
| UGT73C5-R | TTAAGAAGGAGATATACCATGGCTACCAATATTGAGCAGCAGCAGC | SEQ ID NO: 35 |
| UGT73C7-F | TTAAGAAGGAGATATACCATGGATTCACAATTTCAGCAGCTTCACTTTGTTATGA | SEQ ID NO: 36 |
| | TACCC | |
| UGT73C7-R | CTGTTCGACTTAAGCATTAGCTTAATGCTAACCTATCCTTTACTTGTTCAATGAT | SEQ ID NO: 37 |
| | GTCTTG | |
| UGT73C8-F | TAAGAAGGAGATATACCATGGGTTCAGAATCTCAAGTGCAGCTTCAC | SEQ ID NO: 38 |
| UGT73C8-R | GTTCGACTTAAGCATCACTTCCTTTCTTTCAAAATGTCTTGGATTAGTAACGTC | SEQ ID NO: 39 |
| UGT73C9-F | TAAGAAGGAGATATACCATGGCCAACTTAGCTCAAAACCTTCATTTTGTCTTGC | SEQ ID NO: 40 |
| UGT73C9-R | TTCTGTTCGACTTAAGCATCATTTACTTGTCCCATGGGCCATAATTTCTTGAATT | SEQ ID NO: 41 |
| | AGGTG | |
| UGT73C10-F | TAAGAAGGAGATATACCATGTCCCGAATCAGCGGTC | SEQ ID NO: 42 |
| UGT73C10-R | TGTTCGACTTAAGCATCAAACATTTTCTTGATTGTTCAATTTCCGG | SEQ ID NO: 43 |
| UGT73C11-F | TAAGAAGGAGATATACCATGGGAACTATTGCAAACGGAGAAATTGCCC | SEQ ID NO: 44 |
| UGT73C11-R | GTTCGACTTAAGCATCAACAAGTGGTAATTGATCTAATTTCTGAAATGAATTCGT | SEQ ID NO: 45 |
| | CG | |
| UGT73AH1-F | AAGAAGGAGATATACAATGGATTCTCAATTTCAACAATTGCATTTTG | SEQ ID NO: 46 |
| UGT73AH1-R | CAGCAGCCTAGGTTAATttaAGACAAAGCTAATCTATCTTTAACTTGTTCAATTA | SEQ ID NO: 47 |
| | TATCTTG | |
| 28a-R | GTTGAAATTGAGAATCCATggtatatctccttcttaaagttaaacaaaattattt | SEQ ID NO: 48 |
| | ctagaggg | |
| 28a-F | GATAGATTAGCTTTGTCTtaagaattcgagctccgtcgacaag | SEQ ID NO: 49 |
| pETDuet-R | GTATATCTCCTTCTTAAAGTTAAACAAAATTATTTCTAGAGGGG | SEQ ID NO: 50 |
| pETDuet-F | TGCTTAAGTCGAACAGAAAGTAATCGTATTG | SEQ ID NO: 51 |

### Example 3 Induced Expression of Glucosyltransferase

With a strain BL21 (DE3)Δpgi/pETDuet-1 transformed with an empty vector pETDuet-1 as a control, the series of recombinant E. coli constructed in Example 2 were subjected to streak culture on an LB plate containing ampicillin with a concentration of 50 μg/mL at 37° C. for 12 h, respectively. Single bacterial colonies were selected, transferred into 5 mL of an LB liquid culture medium containing ampicillin with a concentration of 50 μg/mL and subjected to shaking culture at 220 rpm at 37° C. for 12 h. The single bacterial colonies were transferred into 25 mL of a TB liquid culture medium containing ampicillin with a concentration of 50 μg/mL at an inoculation amount of 1% by volume and subjected to shaking culture at 220 rpm at 37° C. until an OD₆₀₀ value was 0.6-0.8. Isopropyl thiogalactoside (IPTG) was added to a final concentration of 0.5 μmol/L and subjected to shaking culture continuously at 220 rpm at 16° C. for 20 h.

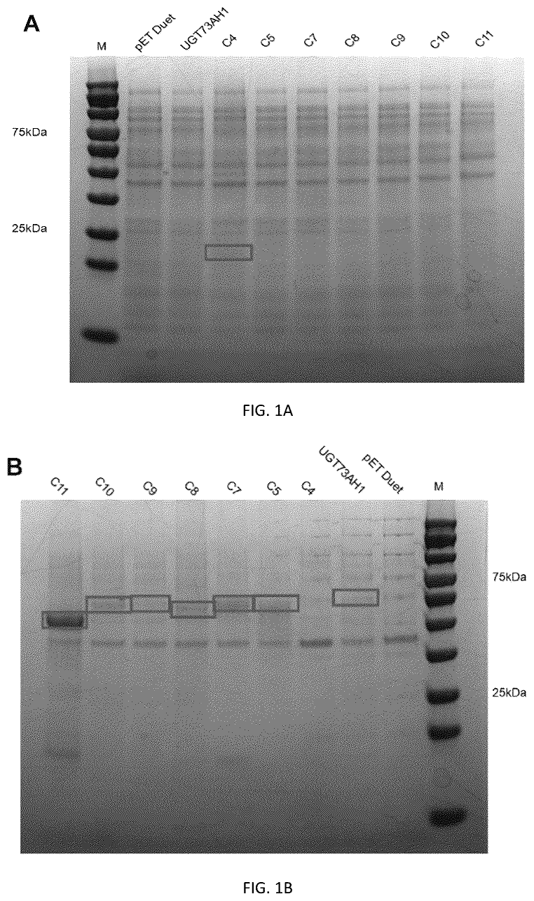

1 mL of a bacterial solution was sucked to determine the final OD₆₀₀ value. 1 mL of the bacterial solution was centrifuged at 5,000×g for 1 min to collect bacteria, and the bacteria were resuspended with 1 mL of a 0.1 M PBS buffer solution with a pH value of 7.4, centrifuged at 5,000×g for 1 min and then washed to remove the residual culture medium. The bacteria were resuspended with a 0.1 M PBS buffer solution with a pH value of 7.4 to control the OD₆₀₀ value of a final resuspended bacterial solution at 5. The bacteria were crushed with an ultrasonic crusher. After completion of crushing, a crushed solution was collected and centrifuged at 12,000×g for 2 min, and a supernatant, namely, a crude enzyme solution of glucosyltransferase, was collected. SDS-PAGE results are shown in FIG. 1.

### Example 4 Production of Asiaticodiglycoside by Induced Fermentation of Recombinant E. coli

With a strain BL21 (DE3)Δpgi/pETDuet-1/pET28a transformed with an empty vector as a control, the series of recombinant E. coli BL21 (DE3)Δpgi/pETDuet-1-UGT73C1/pET28a-UGT73AH1 to BL21 (DE3)Δpgi/pETDuet-1-UGT73C11/pET28a-UGT73AH1 constructed in Example 2 were subjected to streak culture on an LB plate containing kanamycin with a concentration of 50 μg/mL and ampicillin with a concentration of 50 μg/mL at 37° C. for 12 h, respectively. Single bacterial colonies were selected, transferred into 5 mL of an LB liquid culture medium containing kanamycin with a concentration of 50 μg/mL and ampicillin with a concentration of 50 μg/mL and subjected to shaking culture at 220 rpm at 37° C. for 12 h. The single bacterial colonies were transferred into 25 mL of a TB liquid culture medium containing kanamycin with a concentration of 50 μg/mL and ampicillin with a concentration of 50 μg/mL at an inoculation amount of 1% by volume and subjected to shaking culture at 220 rpm at 37° C. until an OD₆₀₀ value was 0.6-0.8. Isopropyl thiogalactoside (IPTG) was added to a final concentration of 0.5 μmol/L, asiatic acid was added as a substrate after 6 h to reach a final concentration of 200 mg/L, shaking culture was performed continuously at 220 rpm at 30° C. for 48 h, and samples were collected and detected at 24 h.

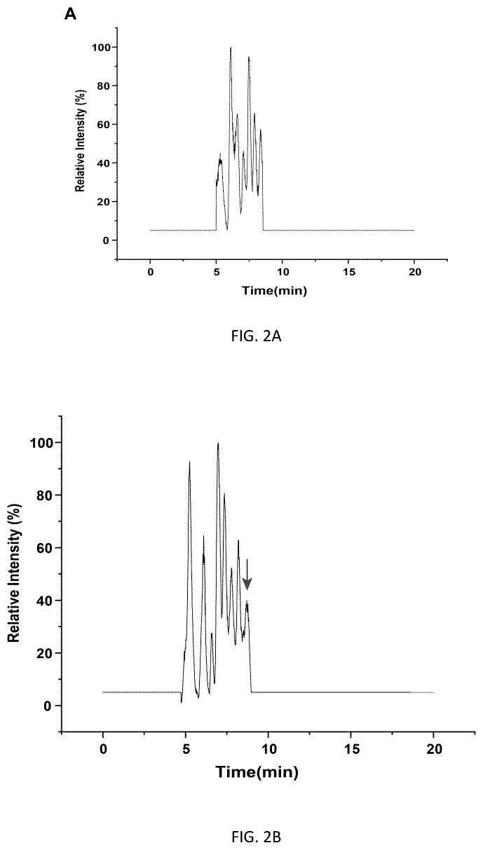

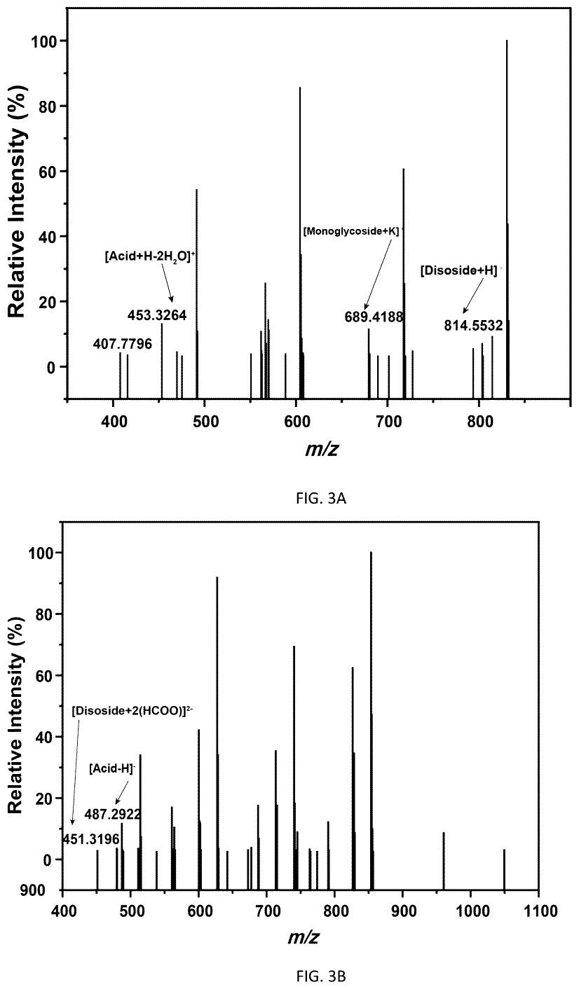

After completion of fermentation, 2 mL of a fermentation solution was taken, added into an equal volume of methanol, violently shaken and uniformly mixed, followed by centrifugation at 14,000 r/min for 10 min. A supernatant was taken and filtered with a 0.22 μm organic phase filter membrane, and a product was detected by UPLC-IT-TOF/MS of Shimadzu. As can be seen from mass spectrograms in FIG. 2A, FIG. 2B, FIG. 3A and FIG. 3B, when UGT73AH1, UGT73C7 and UGT73C8 are transferred into the strain BL21 (DE3)Δpgi at the same time, a new peak different from that of the blank control appears at 7.92 min, and mass spectrum data same as that of asiaticodiglycoside are obtained, determining that asiaticodiglycoside is produced.

### Example 5 Production of Asiaticodiglycoside by a Reaction of a Crude Enzyme Solution

With a strain BL21 (DE3)Δpgi/pETDuet-1/pET28a transformed with an empty vector as a control, the series of recombinant E. coli BL21 (DE3)Δpgi/pETDuet-1-UGT73C1/pET28a-UGT73AH1 to BL21 (DE3)Δpgi/pETDuet-1-UGT73C11/pET28a-UGT73AH1 constructed in Example 2 were subjected to streak culture on an LB plate containing kanamycin with a concentration of 50 μg/mL and ampicillin with a concentration of 50 μg/mL at 37° C. for 12 h, respectively. Single bacterial colonies were selected, transferred into 5 mL of an LB liquid culture medium containing kanamycin with a concentration of 50 μg/mL and ampicillin with a concentration of 50 μg/mL and subjected to shaking culture at 220 rpm at 37° C. for 12 h. The single bacterial colonies were transferred into 25 mL of a TB liquid culture medium containing kanamycin with a concentration of 50 μg/mL and ampicillin with a concentration of 50 μg/mL at an inoculation amount of 1% and subjected to shaking culture at 220 rpm at 37° C. until an OD₆₀₀ value was 0.6-0.8. Isopropyl thiogalactoside (IPTG) was added into a culture to reach a final concentration of 0.5 μmol/L, and shaking culture was performed continuously at 220 rpm at 16° C. for 20 h.

After completion of culture, 1 mL of the culture was sucked to determine the final OD₆₀₀ value. 1 mL of a bacterial solution was centrifuged at 5,000×g for 1 min to collect bacteria. The bacteria were resuspended with 1 mL of a 0.1 M PBS buffer solution with a pH value of 7.4, centrifuged at 5,000×g for 1 min and then washed to remove the residual culture medium. The bacteria were resuspended with a 0.1 M PBS buffer solution with a pH value of 7.4 to control the OD₆₀₀ value of a final resuspended bacterial solution at 5. The bacteria were crushed with an ultrasonic crusher. After completion of crushing, a crushed solution was collected and centrifuged at 12,000×g for 2 min, and a supernatant, namely, a crude enzyme solution of glucosyltransferase, was collected.

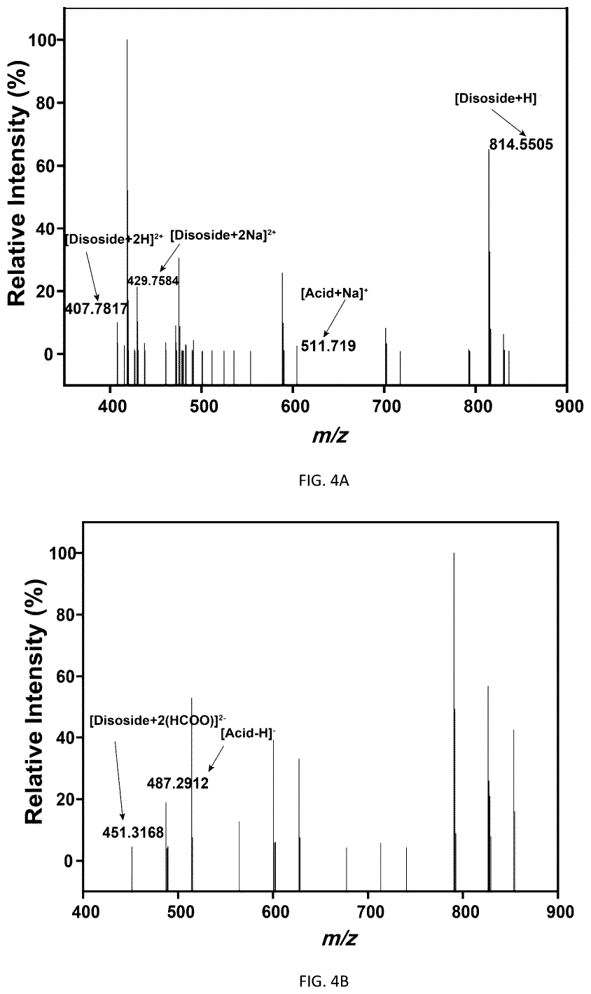

The volume of the total enzyme reaction system was 500 μl, the volume of the crude enzyme solution was 100 μl, the final concentration of the added UDPG was 1 g/L, the final concentration of asiatic acid added as a substrate was 200 mg/L, and the system was supplemented with a PBS buffer solution to 500 μl to carry out a reaction at 37° C. for 6 h. After completion of fermentation, an equal volume of methanol was added, violently shaken and uniformly mixed, followed by centrifugation at 14,000 r/min for 10 min. A supernatant was taken and filtered with a 0.22 μm organic phase filter membrane, and a product was detected by UPLC-IT-TOF/MS of Shimadzu. As can be seen from mass spectrograms in FIG. 4A and FIG. 4B, when UGT73AH1, UGT73C7 and UGT73C8 are transferred into the strain BL21 (DE3)Δpgi at the same time, a new peak different from that of the blank control appears at 7.92 min, and mass spectrum data same as that of asiaticodiglycoside are obtained, determining that asiaticodiglycoside is produced.

### Example 6 Screening of Rhamnosyltransferases with Potential Functions

Through BLAST comparison between data of a transcriptome of Centella asiatica and rhamnosyltransferases that have known functions and are derived from 50 different sources in documents, results show that 5 isoenzymes are basically homologous to genes of the rhamnosyltransferases that have known functions in documents, and the E value is 0. Meanwhile, according to KEGG enrichment analysis, 5 rhamnosyltransferases in the transcriptome of Centella asiatica can be annotated.

TABLE 3
Rhamnosyltransferases with high similarity

| | | Functional | Functional |
|---|---|---|---|
| | | annotation of | annotation |
| Transcriptome gene ID | Number | KEGG | of GO |
| evm.model.CM025780.1.1337[mRNA] | RRT1 | K23280 RRT; | ND |
| evm.model.CM025784.1.1860[mRNA] | RRT2 | rhamnogalacturonan I | |
| evm.model.CM025781.1.322 [mRNA] | RRT3 | rhamnosyltransferase | |
| evm.model.CM025784.1.718[mRNA] | RRT4 | [EC:2.4.1.351] | |
| evm.model.CM025785.1.2450[mRNA] | RRT5 | | |

### Example 7 Gene Amplification of Glycosyltransferase and Construction of Recombinant E. coli for Expressing Rhamnosyltransferase

With cDNA obtained by reverse transcription of Centella asiatica as a template, priming pairs (Table 4) used for amplifying sequences of the rhamnosyltransferases screened in Example 6 were designed and subjected to amplification by PCR, respectively by selecting a Primer Star MasterMix high-fidelity pfu enzyme (Takara) under the following conditions: pre-denaturation at 95° C. for 3 min; at an amplification stage, 30 cycles at 95° C. for 15 s, at 60° C. for 15 s and at 72° C. for 1 min; and extension at 72° C. for 5 min. PCR products were purified to obtain target fragments RRT1 to RRT5, meanwhile, a vector pRSFDuet-1 was subjected to amplification by PCR with a primer pair VvRHM-F and VvRHM-R, and a product was purified. The purified fragments RRT1 to RRT5 were recombined with the vector pRSFDuet-1 skeleton by a Gibson assembly method to obtain recombinant vectors, respectively, and the recombinant vectors were transferred into E. coli JM109. The vectors obtained were sent to Shanghai Biotech for sequencing to obtain correctly sequenced recombinant plasmids pRSFDuet-1-RRT1 to pRSFDuet-1-RRT5. Then, the plasmids were transferred into E. coli BL21 (DE3)Δpgi to obtain recombinant E. coli BL21 (DE3)Δpgi/pRSFDuet-1-RRT1 to BL21 (DE3)Δpgi/pRSFDuet-1-RRT5, respectively.

With a synthetic sequence as a template, a primer pair (Table 4) used for amplifying rhamnose isomerase VvRHM derived from Vitis vinifera was designed and subjected to amplification by PCR by selecting a Primer Star MasterMix high-fidelity pfu enzyme (Takara) under the following conditions: pre-denaturation at 95° C. for 3 min; at an amplification stage, 30 cycles at 95° C. for 15 s, at 56° C. for 15 s and at 72° C. for 1 min; and extension at 72° C. for 5 min. A PCR product was purified, a vector pRSFDuet-1 was subjected to amplification by PCR, and a product was purified. A purified fragment VvRHM and the vector pRSFDuet-1 skeleton were recombined by a Gibson assembly method to obtain a recombinant vector, and the recombinant vector was transferred into E. coli JM109. The obtained vector was sequenced by Shanghai Biotech, correctly compared and then transferred into E. coli BL21 (DE3)Δpgi to obtain recombinant E. coli BL21 (DE3)Δpgi/pRSFDuet-1-VvRHM.

TABLE 4
Primers used for constructing expression vectors of glucosyltransferases

| Primer | Sequence (5′-3′) | |
|---|---|---|
| RRT1-F | TAATAAGGAGATATACCATGGAGGTTAGATCCGAGAGTGTACAGTTG | SEQ ID NO: 52 |
| | AGG | |
| RRT1-R | GATTACTTTCTGTTCGATCATCTCGAAGTTTCTGTTGAATTGACGGTAC | SEQ ID NO: 53 |
| | CTAATACG | |
| RRT2-F | TAATAAGGAGATATACCATGTGTGAATTAGATGAGAACAGGGAAGAG | SEQ ID NO: 54 |
| | AGGGAGA | |
| RRT2-R | GATTACTTTCTGTTCGATCATGTAATTCTTAATGGTTCAACCAAGGGTT | SEQ ID NO: 55 |
| | GCAAACACTC | |
| RRT3-F | ATAAGGAGATATACCATGGAGTTTAGATCTGAGAATGCACAGAATAG | SEQ ID NO: 56 |
| | GTGTGATAAACTG | |
| RRT3-R | CGATTACTTTCTGTTCGATTACCGGAACTTCTCAGTTAAATTGCCCGGG | SEQ ID NO: 57 |
| RRT4-F | TAATAAGGAGATATACCATGGGGTGGAAGACATTAGGAGGTGAGAGT | SEQ ID NO: 58 |
| | AG | |
| RRT4-R | TACTTTCTGTTCGATCAGATGATGCTTAGAAGTTCTTCAGATGAAACTT | SEQ ID NO: 59 |
| | CTGAAAATCGC | |
| RRT5-F | AATAAGGAGATATACCATGAAAAGTGTGAGAGTGGAGGGGAGAGGG | SEQ ID NO: 60 |
| RRT5-R | GATTACTTTCTGTTCGATCATTCAAGTGTATATTGTTCAACCAAGGGCT | SEQ ID NO: 61 |
| | GAAAACAC | |
| VvRHM-F | tttaactttaataaggagatataccATGGCGACCCATACCCCG | SEQ ID NO: 62 |
| VvRHM- | CATggtatatctccttTTACTCGAGCGCTTTCACTTCGG | SEQ ID NO: 63 |
| R | | |

### Example 8 Induced Expression of Rhamosyltranserase

With a strain BL21 (DE3)Δpgi/pRSFDuet-1 transformed with an empty vector pRSFDuet-1 as a control, the series of recombinant E. coli constructed in Example 7 were subjected to streak culture on an LB plate containing kanamycin with a concentration of 50 μg/mL at 37° C. for 12 h, respectively. Single bacterial colonies were selected, transferred into 5 mL of an LB liquid culture medium containing kanamycin with a concentration of 50 μg/mL and subjected to shaking culture at 220 rpm at 37° C. for 12 h. The single bacterial colonies were transferred into 25 mL of a TB liquid culture medium containing kanamycin with a concentration of 50 μg/mL at an inoculation amount of 1% by volume and subjected to shaking culture at 220 rpm at 37° C. until an OD₆₀₀ value was 0.6-0.8. Isopropyl thiogalactoside (IPTG) was added to a final concentration of 0.5 μmol/L and subjected to shaking culture continuously at 220 rpm at 16° C. for 20 h.

After completion of culture, 1 mL of a bacterial solution was sucked to determine the final OD₆₀₀ value. 1 mL of the bacterial solution was centrifuged at 5,000×g for 1 min to collect bacteria, and the bacteria were resuspended with 1 mL of a 0.1 M PBS buffer solution with a pH value of 7.4, centrifuged at 5,000×g for 1 min and then washed to remove the residual culture medium. The bacteria were resuspended with a 0.1 M PBS buffer solution with a pH value of 7.4 to control the OD₆₀₀ value of a final resuspended bacterial solution at 5. The bacteria were crushed with an ultrasonic crusher. After completion of crushing, a crushed solution was collected and centrifuged at 12,000×g for 2 min, and a supernatant, namely, a crude enzyme solution of rhamnosyltransferase, was collected. SDS-PAGE results are shown in FIG. 5.

### Example 9 Production of Asiaticoside by Induced Fermentation of Recombinant E. coli

(1) Construction of Recombinant E. coli for Producing Asiaticoside

Glucosyltransferase UGT73C7 with an amino acid sequence shown in SEQ ID NO: 13 was linked between XbaI and EcoRI restriction enzyme cutting sites of a vector pETDuet-1, glucosyltransferase UGT73AH1 with an amino acid sequence shown in SEQ ID NO: 19 was linked between NdeI and XhoI restriction enzyme cutting sites of the vector pETDuet-1 to construct a recombinant vector, and the recombinant vector was transferred into E. coli JM109. The obtained vector was sequenced by Shanghai Biotech to obtain a correctly sequenced recombinant plasmid pETDuet-1-UGT73AH1-UGT73C7.

Glucosyltransferase UGT73C8 with an amino acid sequence shown in SEQ ID NO: 14 was linked between XbaI and EcoRI restriction enzyme cutting sites of a vector pETDuet-1, glucosyltransferase UGT73AH1 with an amino acid sequence shown in SEQ ID NO: 19 was linked between NdeI and XhoI restriction enzyme cutting sites of the vector pETDuet-1 to construct a recombinant vector, and the recombinant vector was transferred into E. coli JM109. The obtained vector was sequenced by Shanghai Biotech to obtain a correctly sequenced recombinant plasmid pETDuet-1-UGT73AH1-UGT73C8.

The recombinant plasmid pETDuet-1-UGT73AH1-UGT73C7 or pETDuet-1-UGT73AH1-UGT73C8 was transferred into the series of recombinant E. coli constructed in Example 7 to obtain recombinant E. coli BL21 (DE3)Δpgi/pRSFDuet-1-RRT1/pETDuet-1-UGT73AH1-UGT73C7 to BL21 (DE3)Δpgi/pRSFDuet-1-RRT5/pETDuet-1-UGT73AH1-UGT73C7, BL21 (DE3)Δpgi/pRSFDuet-1-RRT1/pETDuet-1-UGT73AH1-UGT73C8 to BL21 (DE3)Δpgi/pRSFDuet-1-RRT5/pETDuet-1-UGT73AH1-UGT73C8, BL21 (DE3)Δpgi/pRSFDuet-1-VvRHM-RRT/pETDuet-1-UGT73AH1-UGT73C7, and BL21 (DE3)Δpgi/pRSFDuet-1-VvRHM-RRT/pETDuet-1-UGT73AH1-UGT73C8.

### (2) Production of Asiaticoside by Shake Flask Fermentation

With a strain BL21 (DE3)Δpgi/pRSFDuet-1/pETDuet-1 transformed with empty vectors pRSFDuet-1 and pETDuet-1 as a control, the series of recombinant E. coli constructed in step (1) were subjected to streak culture on an LB plate containing kanamycin with a concentration of 50 μg/mL and ampicillin with a concentration of 50 μg/mL at 37° C. for 12 h, respectively. Single bacterial colonies were selected, transferred into 5 mL of an LB liquid culture medium containing kanamycin with a concentration of 50 μg/mL and ampicillin with a concentration of 50 μg/mL and subjected to shaking culture at 220 rpm at 37° C. for 12 h. The single bacterial colonies were transferred into 25 mL of a TB liquid culture medium containing kanamycin with a concentration of 50 μg/mL and ampicillin with a concentration of 50 μg/mL at an inoculation amount of 1% by volume and subjected to shaking culture at 220 rpm at 37° C. until an OD₆₀₀ value was 0.6-0.8. IPTG was added to a final concentration of 0.5 μmol/L, asiatic acid was added as a substrate after 6 h to reach a final concentration of 200 mg/L, shaking culture was performed continuously at 220 rpm at 30° C. for 48 h, and samples were collected and detected at 24 h.

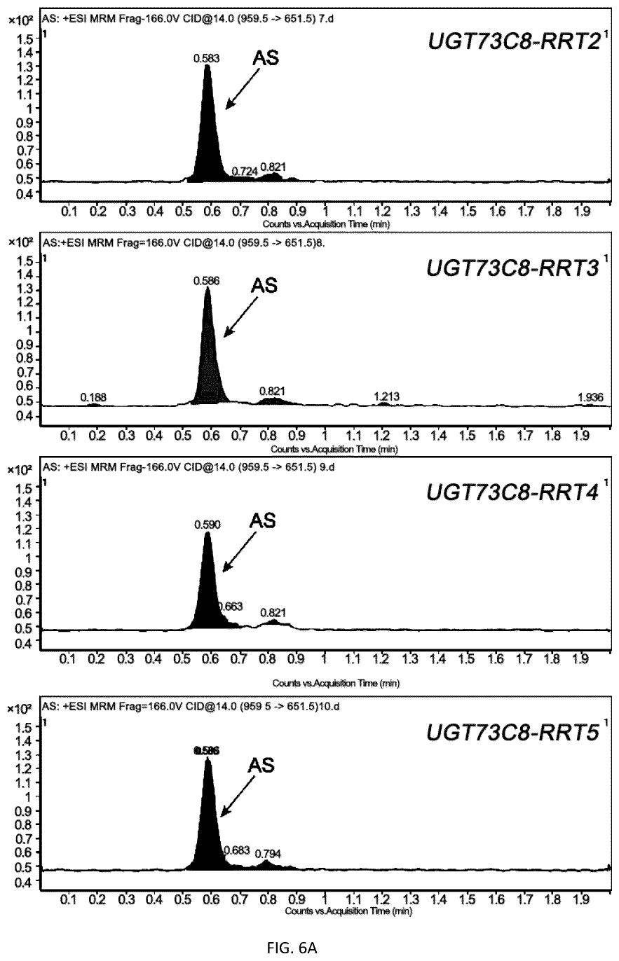

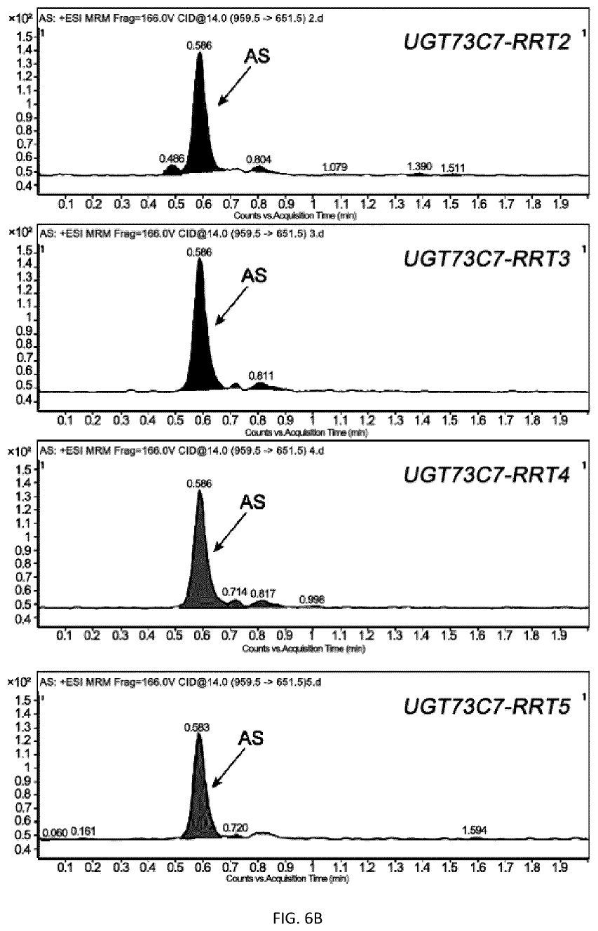

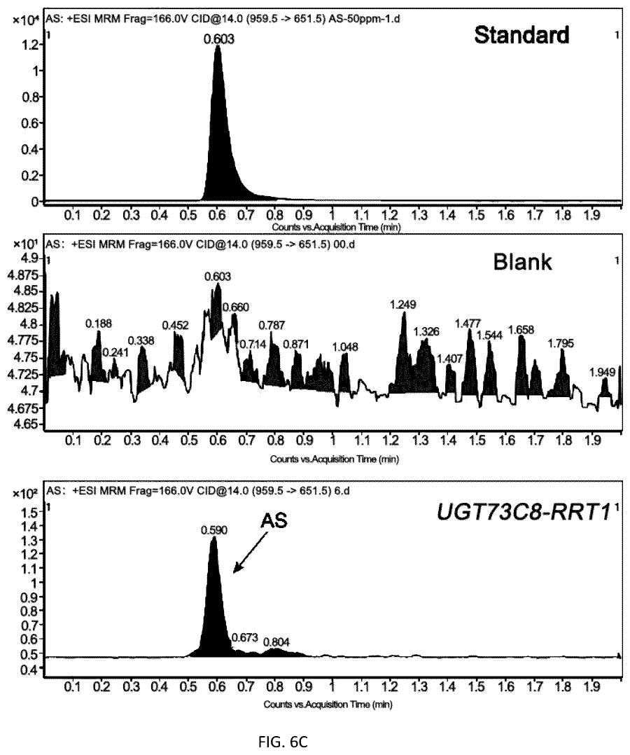

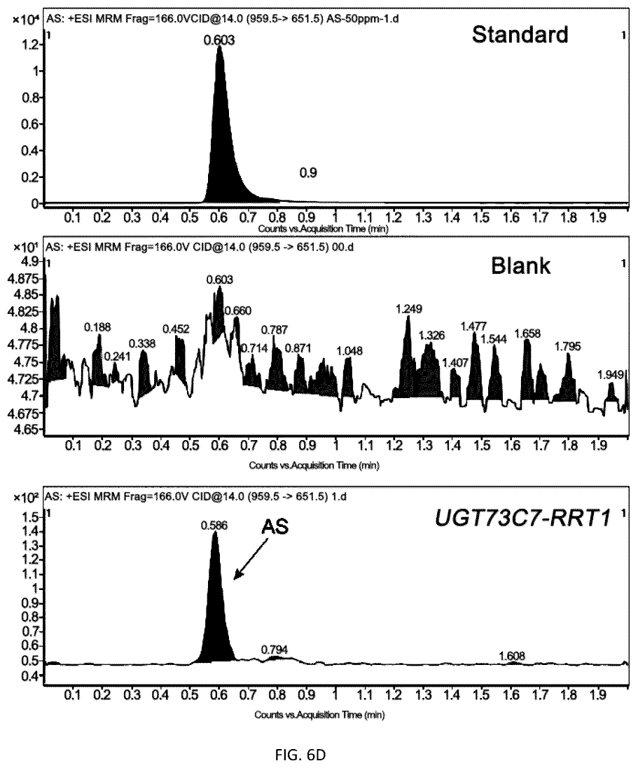

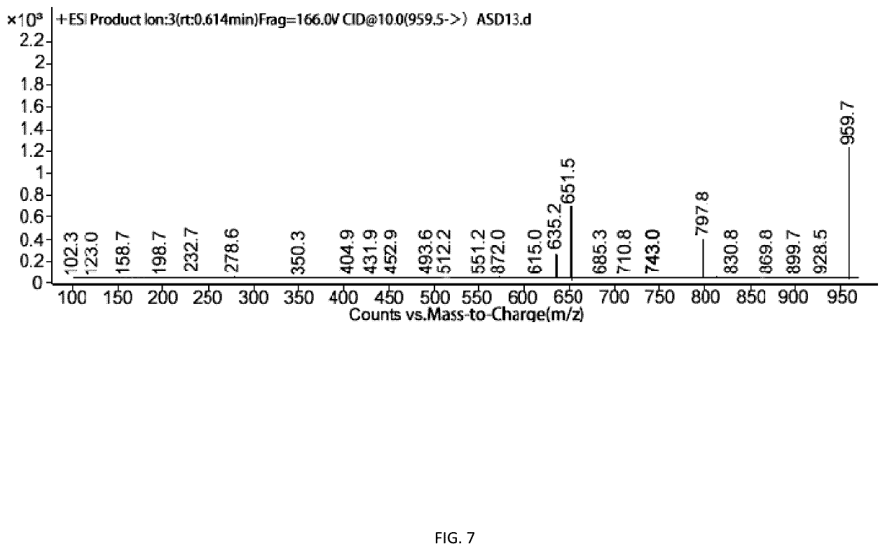

After completion of fermentation, 2 ml of a fermentation solution was taken, added into an equal volume of methanol, violently shaken and uniformly mixed, followed by centrifugation at 14,000 r/min for 10 min. A supernatant was taken and filtered with a 0.22 μm organic phase filter membrane, and a product was detected by UPLC-IT-TOF/MS of Shimadzu. As can be seen from mass spectrograms in FIGS. 6A-D and FIG. 7, the UGT73AH1 and the glucosyltransferase (UGT73C7 or UGT73C8) undergo co-expression with the 5 rhamnosyltransferases screened in Example 6 to prepare asiaticoside by fermentation, respectively, and a unique new peak appears at 0.596 min, which is consistent with a characteristic ion flow of an asiaticoside standard product, determining that the substance is asiaticoside.

Although the present disclosure has been disclosed as above through exemplary examples, the examples are not intended to limit the present disclosure. For any person familiar with the art, various changes and modifications can be made without departing from the spirit and scope of the present disclosure. Therefore, the protection scope of the present disclosure should be as defined in the claims.

What is claimed is:

1. Recombinant Escherichia coli, wherein an E. coli is used as a host and the recombinant E. coli overexpresses glucosyltransferase with the amino acid sequence set forth in any one of SEQ ID NO: 10 to SEQ ID NO: 17 and glycosyltransferase UGT73AH1.

2. The recombinant E. coli according to claim 1, wherein the recombinant E. coli is obtained by knocking out a gene pgi for encoding a glucose phosphate isomerase protein on a genome.

3. The recombinant E. coli according to claim 2, wherein the gene pgi for encoding the glucose phosphate isomerase protein has the nucleotide sequence set forth in SEQ ID NO: 18.

4. The recombinant E. coli according to claim 2, wherein the glycosyltransferase UGT73AH1 is derived from Centella asiatica (L.) Urban.

5. The recombinant E. coli according to claim 1, wherein E. coli BL21 (DE3) is used as a host.

6. The recombinant E. coli according to claim 1, wherein the recombinant E. coli overexpresses rhamnosyltransferase with an amino acid sequence set forth in any one of SEQ ID NO: 25 to SEQ ID NO: 29.

7. The recombinant E. coli according to claim 6, wherein E. coli BL21 (DE3) is used as a host.

8. A method for producing asiaticodiglycoside, comprising inoculating a seed liquid of the recombinant E. coli according to claim 1 into a fermentation culture medium, performing shaking culture until an OD₆₀₀ value is 0.6-0.8, adding isopropyl thiogalactoside, adding asiatic acid with a final concentration of 150-250 mg/L after 4-8 hours, and then performing shaking culture continuously for 40-60 hour;
or with glucosyltransferase with the amino acid sequence shown in any one of SEQ ID NO: 10 to SEQ ID NO: 17 and glycosyltransferase UGT73AH1 derived from Centella asiatica (L.) Urban as a catalyst, carrying out a reaction in a reaction solution containing uridine diphosphate glucose (UDPG) as a glycosyl donor with a final concentration of 0.8-1.2 g/L and asiatic acid as a substrate with a concentration of 150-250 mg/L at 35-38° C. for 4-10 hours.

9. The method according to claim 8, wherein the fermentation culture medium comprises 15-25 g/L of glucose, 4-6 g/L of glycerol, 14-18 g/L of K₂HPO₄·3H₂O, 1-3 g/L of KH₂PO₄, 20-30 g/L of yeast powder and 10-15 g/L of peptone.

10. A method for synthesizing asiaticoside, comprising producing asiaticoside by fermentation with the recombinant E. coli according to claim 6 as an original strain and asiatic acid as a substrate.

11. The method according to claim 10, wherein the method comprises inoculating a seed liquid of the recombinant E. coli according into a fermentation culture medium, performing shaking culture until an OD₆₀₀ value is 0.6-0.8, adding isopropyl thiogalactoside, adding asiatic acid with a final concentration of 150-250 mg/L after 4-8 hours, and then performing shaking culture continuously for 40-60 hours.


SEQUENCE LISTING (69 sequences)

SEQ ID NO: 1 - DNA, 2739 bp, synthetic construct
  source 1..2739: mol_type = other DNA
atgaccagcagtcagctgaaagaactgcattttattttcatcccgctgctgagtccgggtcatgttatgccgatggttgatattgcacgtctgtttgcccagcatggtattaccgtgaccattattagcaccccgctgaataccgcacgttttaaaaccattgtggatcgtgcagtggtggaaagcggtctgcgtattcagattgttgatctggattttccggccgccgaagccggtttaccggaaggttgtcagaatatggatagcattagtcgtgatatgatcaccaatttcttcaccgccagcagtatgctgcagcagccgttagaagatattctggaaaatctgagtccgcgtccgagctgtattattagtggtaaaaatctggcgtggaccgtggaaaccgccctgaaattcaaaattccgcgcctgtttttcgatggtaccggttgttttagtttcgcctgtgcaaatcatctgcagaatagtgaagaattctggcgtgttagtagtaaaaccgaaccggtgctggtgccgggcttagcacatgatattgttctgaccaaagcacagctgccggaagcactgaatccgcatggtagtggcgatctgcatgcagttcgtgaaaaaatggttaatgcagaaagcatcgccgatggcattattgttaatagtttcgaagagctggagccggattatgttaaagattataagcgtgtgaagggcggcaatgtttggtgtattggtccggtgagcgcatgcaataagctgaatattgataaggccgaacgtggtaatctggattgcgcaaaaggtacccattgcctggaatggctggatagccaggaaccgaatagtaccattcatgcatgtctgggtagtattagcggtctgaccgccagccagctggtggagttagcactgggtctggaagccagtaaacgtccgtttatttgggttattcgtagtggtaaacgcagtgaagaactggaaaaatggattgaagaggaaaacttcgaggcccgtaccaaaggtcgtggtctgctgattcgtggttgggcccctcagctgctgattctggctcatagcagcattggtggttttctgacccattgtggttggaatagtaccatcgaaggtctgagtgccggtaaaccgattattacctgtccgctgtttgccgaacagtttattaatgaaaagctggttgtggaggtgctgggtaccggtgttagcgtgggtgtggaagccgcagttgcatggggtctggaagataaaagtggcctggttatgaaacgcgaaaatgtgaaaaaggccattgaaaaagtgctggataagggtaaagagggcgaagaacgtcgcgaacgtgcacgtgaaattggcgaaatggcccgcatggccattgaagaaggtggtagcagctattttaacatgaaaagcctgatccagtacgttatggaacagaatgaactgaatcacggcagtattggcatgctgaaagaacagtttgaaacctttgtgcaggatattaagccgccggcaaattgtattattagcgatagctgcttcccgtggaccaccgaaattagtctgaaatttcatatcccgcgtatcgtttttaacggcccgtgttgttttagcctgctgagcagttataatatcttcaccagccgcgttctggatgaagttgttagtgatagtgaaaccttcgtggtgccgggcctgaatgatcgtgtggaactgaccaaaagtaaactggccggtatgattaatccgcgtgtgaatagcgttatgaaagatctgtttgagcagattatggagagcgaagcagccagctttggtattgtggcaaatacctttgaagagctggaaaccgtgtgtcagcgtgtgcataaaggtagtctgagtcagctgccgaccagtcagatgattgaactgggcctgggcctggaagaaagcaaacgcccttttatttggtgcattcgccatgaaaccgaagaatttggcaaatggattagtaaggagggttatggtgaacgcaccaaaggtcgcggtctgctgatccgtggttgggcacctcaggttctgattctgagccatcaggccattggtggctttctgacccgttgcggctggaatagcaccctggaaggcattagcgcaggtgaacagctgctgacctggcctttctttgcagaacagtttatcaatgaggagttcgtggttaaaatcatccgtaccggtatgcgcattggcgtggaacatccgattgtttttggtgacgaagaacgcgttggcgtgctggcaaaacgcgatgcaattaagaatgccattgaaatgctgatgaacgaagaagaagaggcacaggaacgccgcaaacgcgcaagagaactgggtgaaatggcaagcaaagccaccgaagaaggtggcagcagctatttcaatatgaccatgctgattcaggacattatccagcagcatagcaaaggccgtagcgcagcacatgtgagcgaaaaatttcgtaaatgtcagggtttcaccggcctgattattctggcactgccgtatcgtcattgtaatgaagttgtgtatcgcaaagagatcaagctggaactgcatgaagtttttcgttttgatgaggtggatcatgcactggcaaaagttagcgttgttattagcgaatgcgtgccgggttggatttgggccgaagatattgtgggtccgctgctgaataagtaa

SEQ ID NO: 2 - DNA, 729 bp, synthetic construct
  source 1..729: mol_type = other DNA
atggatgatctctcttctctaaaactgggtgttaaattttttcaagcaaatgaaatgcttcaagaaccactagaaaaactgatgggagaactggtgccaaggccgagctgcatcgtttcaactagtgctctgccgtggactaatgaggttgcttggaaattcaacatcctaagagacagaatggagaaaccgaggcccactgaacatccagctcgagtttctttgtgcaacaaacgtctttcagacatgtcgaatagagaacttggtttagggctagaagcctcaaatcatccctttatgtggataatccgaaagagtgactattcaccacaattagagaaatggttggaggaatatgggtttgaagagagggtgaaaggaagaggcctaattatccatggttgggcgccacaagttctgatactttcgcatccctctgttggaggatttttaactcattgcggatggaattcaactctagaagcagtttgcgcgggcatgccaatgatcacttggcctgtgtttggtgagcagttttacaatgaaaacttcgtggtcaatatactacagattggtgtccgagttggggttgatgttggattgggatctagggggaagtggagtgttacagtgaaggaatctgcagaaaatgaaagcttgaagaatggacacgtcggcaagttggttacacttaaatgtggaagtccgccgtttgttaagggaaagtaa

SEQ ID NO: 3 - DNA, 1488 bp, synthetic construct
  source 1..1488: mol_type = other DNA
atggctaccaatattgagcagcagcagcagcagcttcactttgtgatggttcctcttatgtcccaaagccatatcattcccttgacagattttgccaaactacttgcgcaacgaggacaaattgtgaccataatctcaactcccctcaatgccatccgttacaaacccatcattgatcgagccaagatgaacaacctcaatatccaactagttgctctccagtttccgacccgcgaggctggattgcccgaagggtgtgaaaacatggatgaactctcttctctgaagtttggttttaattttttcgaagcgaatgaaatgctccgggatccactagaaaaagtgatgggagaactggtgccaaggcctagctgcatcgtatcaacaactgctctgccgtggactaatgaggttgcttggaaattcaacattcctaggtatggtttcaatactatttcatgcttcagtctagtatgttctaacaagattaagatttccaaggtgcatgaaagtctcacctcggattcagattcatttgtcgtcccggatttaccagacaaaattgagattaaaaggtcccagttaccggaatatacgatgaaaaaaacttcaaacaatgagggaaaaaacatacttgacaaaatgaaacccacggagcatcccgcacgagggctgttagtgaatacttttgaggagatggagccatcttatgttgaagaatacaggaaacaggaaagtagagtttggtgcattgggtctgtttctttgtccaacaaagatgtttcagatatgtcgaatagaggtaacaaggcctccattgaagaagaagattgcttgaaatggcttgattcaatgaagccatgttctgtcatttatgtttgttttgggagcctttctaatatcccatcctctcaattgatagaacttggtttggggctagaagcctcgaattgccccttcatttggataatccgaaagcgcgactattcaccacaagtagagaaatggctagaagaatacaggtttgaagagagggtgaaaggaagaggcctaattatcctcggttgggcgccacaagtactaatactttcccatccctctgttggaggattcttcactcattgtgggtggaattcaactctagaaggagtttgcgcgggcgtgccgatgatcacttggcctatgtttgctgagcagttttacaatgaaaagtttgtggtcaatgtactaaggactggtgtccgagttggggttgatgttggattgggatctagagaggacgacaactcccaaatcctggtgaagagtgaacaagtgaaggaggtcatagatcagttgatggatggtgaggaggcagaggaaagaagacagagagctcggcaacttaaagaaaaagcaaataaggctattgaaggaggaggatcttcttatttgaacattactatgttcattgaagatgtcaggcaacaaaatttaagtcattcatga

SEQ ID NO: 4 - DNA, 1506 bp, synthetic construct
  source 1..1506: mol_type = other DNA
atggattcacaatttcagcagcttcactttgttatgatacccttaatgtgtcctggccaccttattccactggtggacatggccaaattgcttgctcaacgggccgtcaccgtcaccattgtcaccactccccgaaacgccatccgtttcggggcagccacttctcgggcaatccaatccgggctgcccattcggctcctggaactccggtttccaacgctggaggccggattgcccgaaggttgcgaaaacgttgaccaactctcctcttttgacttctccaaaaactttattgatgcgtcaaaaatgcaacaaggtccattagaaaaaatatttggagacataaagcctagcccaagctgcataatttcggacaagcatatcatgtggaccgctaaaactgctaagaaatttcaaattccatggattgtgtttgatggaatgagctgtttcaatcagttgtgtacagagatattacacactacaaaagtacacgaaaacgtgtccgagtcggaaccctttgttgtccctggtttgcctgatgttattgagttcaagagagcccaactccccggattgtttaatccggggagcaatccaggttcagcgaatgtaaatgatattcgcgaacaaataagggcaactgaagttggagcttatggggttgtgatcaatagttttgaagagttggaacacgattatgttagagaatttaagaagattaaagaaggtaaagtttggtgtgtaggtccattatcattatgcaatactgatgacatagataaggctcagagaggaaacaatgtagccatggatcaacagcataaatgtttacaatggcttgatgcagagaaaaatggttctgtaatatatgtttgtcttggaagcctaagcagccttagtaacctcagtggtgcacagattatagagcttgctttaggcttggaaatgtctggatatccatttatatgggcgctgaggcctgggattaaacataaagagattgagaaatgggtgatagaagaagggtttgaagaaagagttaaaggcagaggaatgttgatctggggttgggcaccacaagtgcttatattgtcacaccgcgctattgggggattcttgacacattgtggatggaattcgacaattgaagggatatgtgctggtgttccgatgatcacatggcctttgttcgcggagcagtttgttaatgagaagtttttagtgcaggttgtgaagactggtgtgagagttggggttgaggctgttgtggatatgggttataaagaagagtttggggtgaaagtgaagagggaggatgttaaaaatgctgtagagtgcatcatgtctgaagatgaggaaggggaatatagaaggaacagagccaagaagcttgcagagatggctgtgaaagcagttgaagagggtggctcttctaatcttaatatgacattgctgattcaagacatcattgaacaagtaaaggataggttagcattaagctaa

SEQ ID NO: 5 - DNA, 1488 bp, synthetic construct
  source 1..1488: mol_type = other DNA
atgggttcagaatctcaagtgcagcttcactttgtcatgatacccttaatgtgtccaggccaccttattccactggtagacatggccaaattgctagcacaactggccgtgaccgtcactattgttaccactccccggaatgccatccgttttggtgcagccacggctcgtgccattgcatctggcctctccattaggcttcttgaactcaggtttccggcagtggaggccggattgccagatgggtgcgaaagcgtagatgatctcccctcttttgacctgacagcaaatttttttgtggcaacaaaaatgatgcaaaagccactagagaacatgttccaagacattaagccaagtccaagctgcataatctcggacaagcttgtggtttggacttctgaaactgctaaaaagtttaacattccttggattatatttgatggaatgagctgttttacacagttgtgtacagatagaatacacacttcaaaagttcatgagactgtatccgagacagaattctttgttgtcccagatttgcctgatgttattgaattcaagaaagcccaactgcccggattgttcaattcagggaacaatccaggttcagtaaatatgaaaaatcgagaagagattagagcagctgaaataggagcttatggggttgttatcaacagttttgaagagttggaacatgcttatgtgaaggaatttaagaagatcagcggaaataaagtttggtgtataggcccattgtcattatgcaataatgatgacatggataaggctcagagaggaaacaatatcatcactgatcatcacctcaaatgcttacaatggcttgatactcaaagaactgactctgtaatatatgcttgtctcggaagcctaagcagccttagtaacctcagttgcgagcagattattgagcttgctttaggcttagaagcttcggggcatccatttatatgggcgctgagggtggggagtaaacacgaaaagatagagaaatggatatcagaagaggggtttgaagaaagaattgaaggcaggggaattctgatccgcggttgggcgccacaagtacttatattgtcacacactgcaataggaggattcttgacacattgcggatggaattcaacagttgaagggatctgcgctggcgtaccaatgatcacatggcctttgttcgcggagcagttttttaatgagaagtttttggtgcaagtggtggagactggtgtgagaattggggttgatgtaattgtgacaatggttgaggaggaaagggttggggtgaaagtgaagagtggggatgttaaaaaggccatagagcgcataatgtgtggaggagaagaaggggaaaatagaaggaaaagagccaaggagcttgcaaagatggctgtggaggcagtggaagagggaggatcttcttaccttaatatgacgttactaatccaagacattttgaaagaaaggaagtga

SEQ ID NO: 6 - DNA, 1482 bp, synthetic construct
  source 1..1482: mol_type = other DNA
atggccaacttagctcaaaaccttcattttgtcttgcttcctttcatggctcaaggccatatcattcctatggtggacatagccaaattgcttgcgcgacgcggcacaattgttaccatcatcagcacacctgttaatgccacccggttcaaagcaactgtcactcgctcagtacaatcgggccttcaaatccgcatggttgaactctattttccaagtgaggaagtaggactgccaaaagggtgtgaaaatatagacatgttgggggcatcaaatttgtctggaaatttttttgaagcaacttttatgctccgagaagctgtagaaaattttcttagagatgcaaatccatccccaagttgcataatctccgatatgtgtttcccatggacaactgatgttgctcaaaagtttcacattccacgacttattttccatggaccatcttgtttttctctcttgtctatgcatcttcttcatacttccaaggttcttgatagactagctccggattctgagaaacttgtattgcctggcctacccgacagaattgaaatggctacggctacgtttgtaggtttgatcaaaccttcgggctcaattcaagattcgtctcccatgaaagacttttttgataagattattgagtccgaaaaggcatcattcggggtagtggctaatacttttgaagagttggagccagagtatgtcaaagagtttgtaagagtaaagggtactaaactttggagtattggacctgtttcattgtgcaacaaagacaacgatgaaaaggtcgagagaggtaacaaaacttcgattaatgaagacgagtgcttgaaatggctcgattcacatgaaccaggctcagtggtgtatgtttgtttgggaagcttatcaaagctagcaacttcccaattgatagaacttgggttgggactagaagcatcgaatgtaccatttatttggtgtataagaagagaaacagaaggatttaggacgtgggctttgcaagaaaattttgaagaaagaatcaaagggagaggccttttgatcaaggggtgggcaccccaagttttgatactgtcacaccaagctgttggaggatttctaacacattgtgggtggaactcgacgttggagggaatttgtgctggcattcaaatgctgacatggcctttgtttgctgaccagttccttaatgaggctttagtagtgcagattctaaggacaggaatgaaggtgggtgtggagagtcctgtcatttttggagaagaagagagtgttggagtgttgatagaaagagatgctgttaaattggctatagacaggctcatgaatcatgaagatgaggggaaggagagaaggaaaagagttaaagaacttggggaaatggctaaaagagcaatggaggaagatggttcttcttaccttaacatgacacacctaattcaagaaattatggcccatgggacaagtaaatga

SEQ ID NO: 7 - DNA, 1527 bp, synthetic construct
  source 1..1527: mol_type = other DNA
atgtcccgaatcagcggtccgccaaacccagtgtcccaaattgattgtccaccaatgtcattgactcaaaaacttcactttgtcctagtcccattactggctcaaggccacatgatcccaatgattgacatggccaggctgctagcgcaacatggcgtcgtggtaagtctggtcaccacccctctcaacgccttgagatttgcgacagcaattcatcgcgcaaggacttctgggcttaaaattcagctcatagaaatcccattcccctggcaagaagtggggcttccatcaggatatgaaaatcttgacagccttccttcgagggacctcataggaaaattttatagtgcacttcatatgttgcaacagccactagaacaacatcttcaacagcaaatgccacctccaagctgcataatttcggacaaatgtctttcctggacatctgagattgccaaaaagtttgaggttccaagactggtttttcatgggatgtgctgtttttcactactaagttcacataatgttaggcttcataatgctcacttgtctgtcacttcagactctcaaccttttgtggttccggggatgccccaaagggttgaaataacaaaagcccagttgccaggagcatttgttaaacttccggatttggacgatattcgtgacagaatgcgtgaggctgaatcaagtgcttatggagttgtggttaacagtttcactgagttggaaaatgggtgtgcgaaaaggtacaaaaaggccatcggtaaaaaggtttggtgcattggaccagtgtcactctgtaataaagaaaatctggacaagtttgagagaggaaacaaggcctcaattgatgagaagagctgcacagagtggcttgattccatgaaaccaaagtctgtgatctatgcttgtttgggcagtcaatgccggctcgttccagcacagctgatggaacttgggttggcactggaatcatcaaaccaacctttcatctgggtaatcaaagaaggagaaagatttcaagaaatggagaaatggttagtagagcagaagtttgaggagagaatcaaagggagaggacttttgatcaaggggtgggcaccacaggttcttatactttcacatccagcaatcaaggcttttttgacacattgtggttggaactcaaccattgaaggagtttgttttggtgtcccaatgataacatggcctatgtttgcagagcagttcttcaatgaaaaacttatagtggatattcttaagattggtgtcaaagttggggttcaattctctgttagatggggggaggaagagaaagttggtgttgtggttaaaaaagggcaaattgagaaagctatagaaaagattatgaatgggggaggtgaaggagaaagtataaagattagagcaagaaacctttcagaaataggaaggcaggccatggaagatggagggtcctctcactccaacatgtcacagttgattcaagatatccggaaattgaacaatcaagaaaatgtttga

SEQ ID NO: 8 - DNA, 1392 bp, synthetic construct
  source 1..1392: mol_type = other DNA
atgggaactattgcaaacggagaaattgcccgaaaacctcacattatgttaatcgcttttccggcgcaaggtcacatgagcccaatgatgcaattcgccaagcgtctagcttccaaaggcacgcgtgtcactgtcgtggctgccgccatcggcattgtcagaagtagtatcgaagaagacacaacgagctccatcaactttgagtacatttcttacaaatttgaagatgatgaaataccagacggtgctaaatcctacatgggctacatgaaactcaaagtcctcaaaagtttgcctggacttgttgaaaaacaaaaattttatgggcaccctgtggaagtcctattgtatgattcgcttcttccatggggattagatttgtgtcgagaacttggagtacgaggaggtccacttttcactcaaagttgtgctgtttgtgccatttactggcacactcatctggggagcctgaggctccccgttgagggtctcacaacggtatccctgtctgcgatgccgttgatggcaattaatgatttgccttcgtccgtgtatgacggtttgtatccgggattgttggagattcttaggaaccagttcgttaatttggagaaagcggattggatattcttcaacacttttgacaagcttgaagatgaggtagcagaatggttgtcaagccagtggcctattaagactatcggaccatcaattccatcaatgttcttagacaaacgattagctgatgacaaagattacggcctcagcctcttcaagccagatgttgaagcttgcatcaagtggttagatgctaaagatgttggctctgtagtctatgtatcttttggcagcttcgtagaccttggccaagaatatatgagcgaactcgcgtggggtttgttaaacagcaacaatttctttttatgggtagttagagaatctgaacagattaagcttccaagtaatttctgtacagaagccgcggagaaaggcctagtagtgagttggtgcccgcagctcgaagttttggctcataagtctgtggggagtttcatgacacattgcgggtggaactcaacaatcgaagcattgagcttaggagtgccaatgctggtaatgcctcaatggacggatcaaacgacgaatgctaaatttattgtggatgtttggcaaactggagttcgagttaaggatattgacaaggagagtgttacaagggaagatattgcagattgcataaaggaggtcatgaatggaacaaaaggggaagagcttaaaacaaatgctgtcaaatggaaggagctggcagtagaggcaatgagtgaaggtgggagctctgacaagaatatcgacgaattcatttcagaaattagatcaattaccacttgttga

SEQ ID NO: 9 - DNA, 1506 bp, synthetic construct
  source 1..1506: mol_type = other DNA
atggattctcaatttcaacaattgcattttgttatgattccattaatgtgtccaggtcacttaattccattagttgatatggctaaattattagcacaaagagctgttactgttactattgttactactcctagaaatgctattaggtttggtgcagctacttctagagcaattcaatctggtttaccaattagattattagaattgagatttccaactttagaagctggtttacctgaaggttgtgaaaatgttgatcaattgtcatcttttgatttttctaaaaattttattgatgcatctaaaatgcaacaaggtccattagaaaagatttttggagatataaaaccatctccttcatgtataattagtgataaacatattatgtggacagctaaaactgctaaaaaatttcaaattccatggattgtttttgatggtatgtcatgttttaatcaattatgtactgaaattttacatacaactaaagttcatgaaaatgtgtctgaatcagaaccttttgttgttccaggtttaccagatgttattgagtttaaaagagctcaattaccaggtttattcaatcctggtagtaatcctggttctgctaatgttaatgacattagagaacaaattagagctacagaagttggtgcatacggtgttgttattaatagttttgaagaattggaacatgactatgttagagaattcaagaaaattaaggaaggtaaagtttggtgtgttggtccattaagtttgtgtaatactgatgatattgataaagctcaaagaggtaataatgttgctatggatcaacaacataaatgtttacaatggttagatgctgaaaagaatggttcagtaatctatgtttgtttgggtagtttatcttcattgtctaacttatcaggtgctcaaattattgaattggcattaggtttagaaatgagtggttacccattcatttgggccttgagaccaggtataaaacataaagaaatagaaaaatgggttattgaagaaggttttgaagaaagagttaaaggtagaggtatgttgatttggggttgggctcctcaagtcttaattttatctcatagagctataggtggatttttaactcattgtggttggaattcaactattgaaggaatttgtgcaggtgttccaatgattacatggccattgtttgctgaacaatttgttaatgaaaaatttttggttcaagttgtaaaaacaggtgttagagttggtgttgaagcagttgttgatatgggttataaagaagaatttggtgttaaagttaaacgtgaagatgttaaaaatgcagttgaatgtataatgagtgaagatgaagaaggtgaatatagaagaaatagagctaaaaaattagctgaaatggctgttaaagctgttgaagaaggtggttctagtaatttgaatatgactttattaattcaagatataattgaacaagttaaagatagattagctttgtcttaa

SEQ ID NO: 10 - protein, 912 aa, synthetic construct
MTSSQLKELHFIFIPLLSPGHVMPMVDIARLFAQHGITVTIISTPLNTARFKTIVDRAVVESGLRIQIVDLDFPAAEAGLPEGCQNMDSISRDMITNFFTASSMLQQPLEDILENLSPRPSCIISGKNLAWTVETALKFKIPRLFFDGTGCFSFACANHLQNSEEFWRVSSKTEPVLVPGLAHDIVLTKAQLPEALNPHGSGDLHAVREKMVNAESIADGIIVNSFEELEPDYVKDYKRVKGGNVWCIGPVSACNKLNIDKAERGNLDCAKGTHCLEWLDSQEPNSTIHACLGSISGLTASQLVELALGLEASKRPFIWVIRSGKRSEELEKWIEEENFEARTKGRGLLIRGWAPQLLILAHSSIGGFLTHCGWNSTIEGLSAGKPIITCPLFAEQFINEKLVVEVLGTGVSVGVEAAVAWGLEDKSGLVMKRENVKKAIEKVLDKGKEGEERRERAREIGEMARMAIEEGGSSYFNMKSLIQYVMEQNELNHGSIGMLKEQFETFVQDIKPPANCIISDSCFPWTTEISLKFHIPRIVFNGPCCFSLLSSYNIFTSRVLDEVVSDSETFVVPGLNDRVELTKSKLAGMINPRVNSVMKDLFEQIMESEAASFGIVANTFEELETVCQRVHKGSLSQLPTSQMIELGLGLEESKRPFIWCIRHETEEFGKWISKEGYGERTKGRGLLIRGWAPQVLILSHQAIGGFLTRCGWNSTLEGISAGEQLLTWPFFAEQFINEEFVVKIIRTGMRIGVEHPIVFGDEERVGVLAKRDAIKNAIEMLMNEEEEAQERRKRARELGEMASKATEEGGSSYFNMTMLIQDIIQQHSKGRSAAHVSEKFRKCQGFTGLIILALPYRHCNEVVYRKEIKLELHEVFRFDEVDHALAKVSVVISECVPGWIWAEDIVGPLLNK

SEQ ID NO: 11 - protein, 242 aa, synthetic construct
MDDLSSLKLGVKFFQANEMLQEPLEKLMGELVPRPSCIVSTSALPWTNEVAWKFNILRDRMEKPRPTEHPARVSLCNKRLSDMSNRELGLGLEASNHPFMWIIRKSDYSPQLEKWLEEYGFEERVKGRGLIIHGWAPQVLILSHPSVGGFLTHCGWNSTLEAVCAGMPMITWPVFGEQFYNENFVVNILQIGVRVGVDVGLGSRGKWSVTVKESAENESLKNGHVGKLVTLKCGSPPFVKGK

SEQ ID NO: 12 - protein, 495 aa, synthetic construct
MATNIEQQQQQLHFVMVPLMSQSHIIPLTDFAKLLAQRGQIVTIISTPLNAIRYKPIIDRAKMNNLNIQLVALQFPTREAGLPEGCENMDELSSLKFGFNFFEANEMLRDPLEKVMGELVPRPSCIVSTTALPWTNEVAWKFNIPRYGFNTISCFSLVCSNKIKISKVHESLTSDSDSFVVPDLPDKIEIKRSQLPEYTMKKTSNNEGKNILDKMKPTEHPARGLLVNTFEEMEPSYVEEYRKQESRVWCIGSVSLSNKDVSDMSNRGNKASIEEEDCLKWLDSMKPCSVIYVCFGSLSNIPSSQLIELGLGLEASNCPFIWIIRKRDYSPQVEKWLEEYRFEERVKGRGLIILGWAPQVLILSHPSVGGFFTHCGWNSTLEGVCAGVPMITWPMFAEQFYNEKFVVNVLRTGVRVGVDVGLGSREDDNSQILVKSEQVKEVIDQLMDGEEAEERRQRARQLKEKANKAIEGGGSSYLNITMFIEDVRQQNLSHS

SEQ ID NO: 13 - protein, 501 aa, synthetic construct
MDSQFQQLHFVMIPLMCPGHLIPLVDMAKLLAQRAVTVTIVTTPRNAIRFGAATSRAIQSGLPIRLLELRFPTLEAGLPEGCENVDQLSSFDFSKNFIDASKMQQGPLEKIFGDIKPSPSCIISDKHIMWTAKTAKKFQIPWIVFDGMSCFNQLCTEILHTTKVHENVSESEPFVVPGLPDVIEFKRAQLPGLFNPGSNPGSANVNDIREQIRATEVGAYGVVINSFEELEHDYVREFKKIKEGKVWCVGPLSLCNTDDIDKAQRGNNVAMDQQHKCLQWLDAEKNGSVIYVCLGSLSSLSNLSGAQIIELALGLEMSGYPFIWALRPGIKHKEIEKWVIEEGFEERVKGRGMLIWGWAPQVLILSHRAIGGFLTHCGWNSTIEGICAGVPMITWPLFAEQFVNEKFLVQVVKTGVRVGVEAVVDMGYKEEFGVKVKREDVKNAVECIMSEDEEGEYRRNRAKKLAEMAVKAVEEGGSSNLNMTLLIQDIIEQVKDRLALS

SEQ ID NO: 14 - protein, 495 aa, synthetic construct
MGSESQVQLHFVMIPLMCPGHLIPLVDMAKLLAQLAVTVTIVTTPRNAIRFGAATARAIASGLSIRLLELRFPAVEAGLPDGCESVDDLPSFDLTANFFVATKMMQKPLENMFQDIKPSPSCIISDKLVVWTSETAKKFNIPWIIFDGMSCFTQLCTDRIHTSKVHETVSETEFFVVPDLPDVIEFKKAQLPGLFNSGNNPGSVNMKNREEIRAAEIGAYGVVINSFEELEHAYVKEFKKISGNKVWCIGPLSLCNNDDMDKAQRGNNIITDHHLKCLQWLDTQRTDSVIYACLGSLSSLSNLSCEQIIELALGLEASGHPFIWALRVGSKHEKIEKWISEEGFEERIEGRGILIRGWAPQVLILSHTAIGGFLTHCGWNSTVEGICAGVPMITWPLFAEQFFNEKFLVQVVETGVRIGVDVIVTMVEEERVGVKVKSGDVKKAIERIMCGGEEGENRRKRAKELAKMAVEAVEEGGSSYLNMTLLIQDILKERK

SEQ ID NO: 15 - protein, 493 aa, synthetic construct
MANLAQNLHFVLLPFMAQGHIIPMVDIAKLLARRGTIVTIISTPVNATRFKATVTRSVQSGLQIRMVELYFPSEEVGLPKGCENIDMLGASNLSGNFFEATFMLREAVENFLRDANPSPSCIISDMCFPWTTDVAQKFHIPRLIFHGPSCFSLLSMHLLHTSKVLDRLAPDSEKLVLPGLPDRIEMATATFVGLIKPSGSIQDSSPMKDFFDKIIESEKASFGVVANTFEELEPEYVKEFVRVKGTKLWSIGPVSLCNKDNDEKVERGNKTSINEDECLKWLDSHEPGSVVYVCLGSLSKLATSQLIELGLGLEASNVPFIWCIRRETEGFRTWALQENFEERIKGRGLLIKGWAPQVLILSHQAVGGFLTHCGWNSTLEGICAGIQMLTWPLFADQFLNEALVVQILRTGMKVGVESPVIFGEEESVGVLIERDAVKLAIDRLMNHEDEGKERRKRVKELGEMAKRAMEEDGSSYLNMTHLIQEIMAHGTSK

SEQ ID NO: 16 - protein, 508 aa, synthetic construct
MSRISGPPNPVSQIDCPPMSLTQKLHFVLVPLLAQGHMIPMIDMARLLAQHGVVVSLVTTPLNALRFATAIHRARTSGLKIQLIEIPFPWQEVGLPSGYENLDSLPSRDLIGKFYSALHMLQQPLEQHLQQQMPPPSCIISDKCLSWTSEIAKKFEVPRLVFHGMCCFSLLSSHNVRLHNAHLSVTSDSQPFVVPGMPQRVEITKAQLPGAFVKLPDLDDIRDRMREAESSAYGVVVNSFTELENGCAKRYKKAIGKKVWCIGPVSLCNKENLDKFERGNKASIDEKSCTEWLDSMKPKSVIYACLGSQCRLVPAQLMELGLALESSNQPFIWVIKEGERFQEMEKWLVEQKFEERIKGRGLLIKGWAPQVLILSHPAIKAFLTHCGWNSTIEGVCFGVPMITWPMFAEQFFNEKLIVDILKIGVKVGVQFSVRWGEEEKVGVVVKKGQIEKAIEKIMNGGGEGESIKIRARNLSEIGRQAMEDGGSSHSNMSQLIQDIRKLNNQENV

SEQ ID NO: 17 - protein, 463 aa, synthetic construct
MGTIANGEIARKPHIMLIAFPAQGHMSPMMQFAKRLASKGTRVTVVAAAIGIVRSSIEEDTTSSINFEYISYKFEDDEIPDGAKSYMGYMKLKVLKSLPGLVEKQKFYGHPVEVLLYDSLLPWGLDLCRELGVRGGPLFTQSCAVCAIYWHTHLGSLRLPVEGLTTVSLSAMPLMAINDLPSSVYDGLYPGLLEILRNQFVNLEKADWIFFNTFDKLEDEVAEWLSSQWPIKTIGPSIPSMFLDKRLADDKDYGLSLFKPDVEACIKWLDAKDVGSVVYVSFGSFVDLGQEYMSELAWGLLNSNNFFLWVVRESEQIKLPSNFCTEAAEKGLVVSWCPQLEVLAHKSVGSFMTHCGWNSTIEALSLGVPMLVMPQWTDQTTNAKFIVDVWQTGVRVKDIDKESVTREDIADCIKEVMNGTKGEELKTNAVKWKELAVEAMSEGGSSDKNIDEFISEIRSITTC

SEQ ID NO: 18 - DNA, 1650 bp, synthetic construct
  source 1..1650: mol_type = other DNA
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 19 - protein, 501 aa, synthetic construct; identical to SEQ ID NO: 13

SEQ ID NO: 20 - DNA, 1497 bp, synthetic construct
  source 1..1497: mol_type = other DNA
atggaggttagatccgagagtgtacagttgaggtgtgataagctgattcccagaactagatttcaagtgtggttcattaggatttgtttcagcattttgatatggacctgtttggttcagctagttgcagttggtgagttgtggcatccatatttgttaagtgggttatcggatcgttcaaatgggtcttctaagctatttataaacaggaatgcagttccgtccttgcctccacttcctccggcaaggaactacacgagtaatggttttctcagggtttcttgcaatgggggtttaaatcaaatgcgtgctgcgatctgtgacatggttgcagtttctcgactcttgaaccttactttggttgttccagagcttgataagacatcattctgggcagatcctagtgattttgaggacatttttgatgtgagacacttcattgattcattgagagatgaagttcgaattgtcaaaaggttaccaaaaaggtttatccaaagatatggatacaggccattagaaatgcctccggtcagctggtccagtgaaaaatactatctgcaacagattctgcctctttttgacaagcataagttgatacatttcaacagaacagatacacggttggcaaacaatatgattccccttgatcttcagaaattcaggtgtcgcgtaaattttcgggcactgagattcactcctcaaattgaggctttgggacagaaattggttcatatacttgaagggaaaggatcatttatagcattacatctcagatacgaaatggatatgttggctttctcgggttgtacacatggatgcacagaggaagaagctgaagagctgaaacgattgaggtatgcatttccttggtggagggagaaagagatagtgtctggagaaaggagatcacaaggtctgtgccctcttacccctgaggaggcagctttagtgctgcgggcactagatattgacaataacacccagatatatattgcatctggtgagatttatggcagcgaaaagagacttgctgtgttaaaggctgcatttccacggattgtgaagaaggaaatgctgctagatgctgatgatttgcgacaattccaaaatcattcatcccaaatggcagctctggacttcatggtatctgttgccagcaatgttttcattcccacttatgacggtaacatggcaaagcttgtggaaggtcatcgcagatattttgggtttaaaaagaccatccaactagatcggcgaagaattgtgcaattgattgatttacatcagaacgggacacttgcatgggatgagttctcagctgctattagattgacacatgaaggaaggacaggacagcccacacgtcgtagagttgatgcagaaaaacccaaagaagaagactacttctatgcgaacccttatgagtgtttttgcgagggaacaagttgtggtaacgtattaggtaccgtcaattcaacagaaacttcgagatga

SEQ ID NO: 21 - DNA, 1530 bp, synthetic construct
  source 1..1530: mol_type = other DNA
atgtgtgaattagatgagaacagggaagagagggagaaacagtggaagattggtatgaagtttgtgggtgaaattaaagaggaaagacaaagtttgatggtttctcatgctcgaaagttgttgtggattgtccgaggaatgacaacagtgttgctttggacttttgttattcgattgttgacaatgggtgagatttggggtccaaggttgttgaagagttggccgtcatgttttaatcctcctccaaatatgttaactgaggctaagatgtcttacattcatcccaaagtttttcatcctcctaagagggtttacaagaacaatggttatcttatggtttcttgcaatggaggcctcaatcaaatgagaggagcaatatgtgacatggttgctattgcaagatacctgaatgtcactcttattgttccagaactggataaagcctcattttgggcagatccaagcgagtttcaggacatatttgatgttgatcatttcataacatctttgagggatgaggttcggattttgaaagagctgccaccacggctcaagagaagagttgagcttggactgttctactcattgccgcctattagttggtctgacatttcctactaccaaaaccagattcttcctctacttcaaaagtacaaagtcgtgcatttaaatagaactgatgctcgacttgctaataatgggttaccgctagagattcagaaattgcgctgcaaagttaattttagtgctctgagattcatgtcccaaatagaggagctaggtagaaagattgtcagcatcttgagggaaaatggcccttttgttgtcctccatcttaggtatgagatggatatgttggctttctctggctgcagtcatggctgcaacagtgaagaggtcaatgaattaacaagaatgagatatgcttacccctggtggaaggaaaaaattattgattcggaccttaaaaggaaagaaggtttgtgtcctttgacacctgaagaaactgctctaatattaacagctttgggtattgatcatgatgttcaaatttatattgctgctggagaaatttatggtggaggacggagactagcaagcttggcagctgcctttccaaatttggtcaaaaaggagaaattgctggatccttcagatttgaagtactttcagaaccactcctcccaaatggctgcactagattatctcgtctccttggagagtgacatatttgtgcctacatatgatggaaacatggctaaagttgttgagggtcatcgtagatatttaggatacaagaagactatctcattggacagaaagcttctagttgatttgatagaccgtcactatgctgggtcactaagctgggacgagtttgcttcaacagtgaaggaaacacatgcagatagaatgggtagtgcaagagaacgactagtgatcccagacagaccgaaggaagaagactatttctttgccaatccacacgagtgtttgcaacccttggttgaaccattaagaattacatga

SEQ ID NO: 22 - DNA, 1509 bp, synthetic construct
  source 1..1509: mol_type = other DNA
atggagtttagatctgagaatgcacagaataggtgtgataaactgcccgggcaggtgattccgaagactaggttacaaatatggttcattagggtttgttccagcattttgatatggacttgtttagttcagctagttacggttggcgagctttggcatccacggttgctggcagggctatccaatcgttcaaatggctcttccaagatttcaacagaggaaagggcggttccgtcgctgcttcctcttcttcctgcaaggaactacactagtaatggttttctcaaagtttcttgcaatggaggtttgaatcaaatgcgtgctgcgatctgtgacatggttacagttgctcgactattaaatcttactttggttgttccagagctggataagacatccttctgggctgatcctagtaatttcgaggatatttttaatgtgagacacttcattgattcattgagagatgaagttcacattgtcaaaaggattccaaaaaaatttatcaggagatatggataccagtcactacaaatgcctcctgttagctggtcgagtgaaaaatactacctggaacagatactgccactttttaacaagcataagttgatacatttcaacagaacagatacacggttggcaaacaatatgatcccccttgatcttcagaaactcaggtgccgtgtcaatttccaggcactgagattcacccctcagattgaggctttgggacagaaattggttgatattcttcaagggaaaggaccatttatagcattgcatctaagatatgagatggatatgctggctttctcaggttgtacacatggctgcacagaggaagaagctgtaaagctaaaacgcttgaggtatgcatttccttggtggagggagaaagagatagtgtctgaagagaggagatcactaggtctatgccctcttactccagaggaggcagttttagtgctgcaagcattaaattttgacaaggacacacaaatatatattgcctctggagagatttatggcagtgaaaggagacttgcagttctacgggcttcatttccacggattgtgaagaaagaaatgttgctagatcccggggaattgcaacaatttcaaaatcattcatcccaaatggctgccctggacttcattgtttccgtcgctagtagtgttttcattcccacatatgatggcaacatggcaaagcttgtggaaggtcaccgcaggtatcttgggttcagaaagaccctccaactagaccgaagaaaaattgtagaattaatcgactcatatcagaacaagacactttcatgggaggatttcgtgggtgccatgagattgacacatgagagaagaacaggacagcccacaggtcgtaaagtcattgcagacaagcccaaagaagaagattatttttatgcaaaccctcacgagtgcctctgtgaaagtccaaattgtgacaatttacaacccccgggcaatttaactgagaagttccggtaa

SEQ ID NO: 23 - DNA, 1500 bp, synthetic construct
  source 1..1500: mol_type = other DNA
atggggtggaagacattaggaggtgagagtagaaaaaggattgagaagttaaggagttcaatggtttctaggtctagaatgaagctgtggatgattagagctactacttcattgttgttgtggacatgtgttgttcagttgacagctttgggagagctgtgggggcctagggttttgaagggttggccttcttgttttactcaggattctgtcatggatattaagatgactccagctcttccagctagagttcttccacccaagagggtttataagaacaatggttatttaatggtttcatgcaatggagggcttaatcagatgcgggctgcgatatgcgacatggttgctattgcaagatatttaaatgttacacttatagttcctgagctggataaaacatctttttgggctgatccaagcgagttccaagacatttttgatgttgaccatttcatttcgtccttgagagatgaggttcgtatactgaaagagcttcctcccaggattaagaggagagtggaactaggaatgttctatacaatggcacccgtcagttggtctgacatctcttactatcataatcagattcttcctctggtacagaagtataaagttgtgcatttgaatagaactgatgctcgccttgctaacaatggccagccacttgagattcagaagctccggtgccgagtaaactttggagctctgagatttactcctcagatagaggagttgggaaggagggtagtcaagcttctaaggcagaatggtccttttctagtacttcatctcagatatgaaatggacatgctagcattttcaggctgtagtcagggttgcaacaatgaagaggtggatgagttgacgcggatgagatatgcatacccatggtggaaagagaagatcataaactctgatttgaaaagaaaggatggtctatgtccgttgacccctgaagaaactgctctcacactaagggcgctggacattgatcatgacatacagatttacattgctgcaggagaaatatatggcggagagaggagaatggctagtcttgctgccgcttatccgaaactggttaggaaggagactcttctgagtcagtcagaccttggatattttaagaatcactcttctcagatggcagcattagattatcttgtttcgttggagagtgatatttttgttcctacatatgacggaaacatggccaaagttgttgaaggtcatcggagattccttgggttgaagaagacaatactattggacagaaagctattagttgatttgatagaccaatataatcatgggtcactgaattgggatgaattctctattgctgttaagaacgctcatgcagaccgcatgggaaatgccaccaaaagattagtgattccagacagacctaaagaagaggactatttctatgcgaatccagaagagtgcttgcgattttcagaagtttcatctgaagaacttctaagcatcatctga

SEQ ID NO: 24 - DNA, 1506 bp, synthetic construct
  source 1..1506: mol_type = other DNA
atgaaaagtgtgagagtggaggggagagggagaaaagagagttggagtatgaagtttgtggatgagaagaaagaggcaaaatcaaggtttatggtttctcggtctcgaatgatgttgtggattatcagaggaatgacgactttgttgctttggacatttgttattcgattattgacaatgggtgaagtttggggtccaaggttgttgaagagttggccttcatgtatgtttactgaggtcaacctgtctgatgctcagcccaaggtttttcagcctcccaagagggtttataagaacaatggctatcttttggtttcttgcaatggaggactcaatcaaatgcgaggagccatttgtgacatggttgctgttgcaagacatctgaatgtcacacttattgtgccagaactggataaaacttcattttgggcagatccaagtgagtttaaggacatatttgatgttgatcattttattgcatccttgagagatgaggtgcgaattttgaaggaactgccaccacggcttaagagaagagttgagcttggactatatcactcattgccgcctattagttggtcggacatttcctactatcataatcagattcttccactactacaaaaatataaagttgttcatttaaatagaaccgatgctcgacttgctaacaatgggttacctctggagatacagaagctgcgttgcagagtaaattttagtgctctgaaattcacttctcaaatagaaaccctaggtaggaaggtagtcagcatcttgaggcaaaatggcccttttgtcgtcctccatctaagatatgaaatggatatgttggctttctccggctgcagccacggctgcaacaacgaagaggtggaacacctaacacgaatgagatatgcttatccctggtggaaggaaaaggttatagattcagacttcaaaaggcgagaaggtttatgccctctgacacctgaagaaactgctcaagtattaactgcattgggcatcaatcgcgatgttcaaatatatatagctgctggagaaatttatggtggagctcgcagaatggcaagtttggctgctgcatttccaaatttggtcagaaaggagaacctgttggatccttcagatttgaggtatttccagaaccactcatcccaaatggctgcactagattatcttgtgtccttgcagagtgacatatttgtaccaacatatgatggaaacatggctaaagttgttgaaggtcatcgcagatacttaggattcaagaagaccatctcattgaacagaaagcttctagtagatttgattgatcgttatcatggcggattgttaagctgggatgacttctcttccatggtgaaggaatctcatgcagatcggatggggagtgccaaacagcgattagtaatcccagatagaccaaaggaagaagactatttctatgccaatccacatgagtgttttcagcccttggttgaacaatatacacttgaatga

SEQ ID NO: 25 - protein, 498 aa, synthetic construct
MEVRSESVQLRCDKLIPRTRFQVWFIRICFSILIWTCLVQLVAVGELWHPYLLSGLSDRSNGSSKLFINRNAVPSLPPLPPARNYTSNGFLRVSCNGGLNQMRAAICDMVAVSRLLNLTLVVPELDKTSFWADPSDFEDIFDVRHFIDSLRDEVRIVKRLPKRFIQRYGYRPLEMPPVSWSSEKYYLQQILPLFDKHKLIHFNRTDTRLANNMIPLDLQKFRCRVNFRALRFTPQIEALGQKLVHILEGKGSFIALHLRYEMDMLAFSGCTHGCTEEEAEELKRLRYAFPWWREKEIVSGERRSQGLCPLTPEEAALVLRALDIDNNTQIYIASGEIYGSEKRLAVLKAAFPRIVKKEMLLDADDLRQFQNHSSQMAALDFMVSVASNVFIPTYDGNMAKLVEGHRRYFGFKKTIQLDRRRIVQLIDLHQNGTLAWDEFSAAIRLTHEGRTGQPTRRRVDAEKPKEEDYFYANPYECFCEGTSCGNVLGTVNSTETSR

SEQ ID NO: 26 - protein, 509 aa, synthetic construct
MCELDENREEREKQWKIGMKFVGEIKEERQSLMVSHARKLLWIVRGMTTVLLWTFVIRLLTMGEIWGPRLLKSWPSCFNPPPNMLTEAKMSYIHPKVFHPPKRVYKNNGYLMVSCNGGLNQMRGAICDMVAIARYLNVTLIVPELDKASFWADPSEFQDIFDVDHFITSLRDEVRILKELPPRLKRRVELGLFYSLPPISWSDISYYQNQILPLLQKYKVVHLNRTDARLANNGLPLEIQKLRCKVNFSALRFMSQIEELGRKIVSILRENGPFVVLHLRYEMDMLAFSGCSHGCNSEEVNELTRMRYAYPWWKEKIIDSDLKRKEGLCPLTPEETALILTALGIDHDVQIYIAAGEIYGGGRRLASLAAAFPNLVKKEKLLDPSDLKYFQNHSSQMAALDYLVSLESDIFVPTYDGNMAKVVEGHRRYLGYKKTISLDRKLLVDLIDRHYAGSLSWDEFASTVKETHADRMGSARERLVIPDRPKEEDYFFANPHECLQPLVEPLRIT

SEQ ID NO: 27 - protein, 502 aa, synthetic construct
MEFRSENAQNRCDKLPGQVIPKTRLQIWFIRVCSSILIWTCLVQLVTVGELWHPRLLAGLSNRSNGSSKISTEERAVPSLLPLLPARNYTSNGFLKVSCNGGLNQMRAAICDMVTVARLLNLTLVVPELDKTSFWADPSNFEDIFNVRHFIDSLRDEVHIVKRIPKKFIRRYGYQSLQMPPVSWSSEKYYLEQILPLFNKHKLIHFNRTDTRLANNMIPLDLQKLRCRVNFQALRFTPQIEALGQKLVDILQGKGPFIALHLRYEMDMLAFSGCTHGCTEEEAVKLKRLRYAFPWWREKEIVSEERRSLGLCPLTPEEAVLVLQALNFDKDTQIYIASGEIYGSERRLAVLRASFPRIVKKEMLLDPGELQQFQNHSSQMAALDFIVSVASSVFIPTYDGNMAKLVEGHRRYLGFRKTLQLDRRKIVELIDSYQNKTLSWEDFVGAMRLTHERRTGQPTGRKVIADKPKEEDYFYANPHECLCESPNCDNLQPPGNLTEKFR

SEQ ID NO: 28 - protein, 499 aa, synthetic construct
MGWKTLGGESRKRIEKLRSSMVSRSRMKLWMIRATTSLLLWTCVVQLTALGELWGPRVLKGWPSCFTQDSVMDIKMTPALPARVLPPKRVYKNNGYLMVSCNGGLNQMRAAICDMVAIARYLNVTLIVPELDKTSFWADPSEFQDIFDVDHFISSLRDEVRILKELPPRIKRRVELGMFYTMAPVSWSDISYYHNQILPLVQKYKVVHLNRTDARLANNGQPLEIQKLRCRVNFGALRFTPQIEELGRRVVKLLRQNGPFLVLHLRYEMDMLAFSGCSQGCNNEEVDELTRMRYAYPWWKEKIINSDLKRKDGLCPLTPEETALTLRALDIDHDIQIYIAAGEIYGGERRMASLAAAYPKLVRKETLLSQSDLGYFKNHSSQMAALDYLVSLESDIFVPTYDGNMAKVVEGHRRFLGLKKTILLDRKLLVDLIDQYNHGSLNWDEFSIAVKNAHADRMGNATKRLVIPDRPKEEDYFYANPEECLRFSEVSSEELLSII

SEQ ID NO: 29 - protein, 501 aa, synthetic construct
MKSVRVEGRGRKESWSMKFVDEKKEAKSRFMVSRSRMMLWIIRGMTTLLLWTFVIRLLTMGEVWGPRLLKSWPSCMFTEVNLSDAQPKVFQPPKRVYKNNGYLLVSCNGGLNQMRGAICDMVAVARHLNVTLIVPELDKTSFWADPSEFKDIFDVDHFIASLRDEVRILKELPPRLKRRVELGLYHSLPPISWSDISYYHNQILPLLQKYKVVHLNRTDARLANNGLPLEIQKLRCRVNFSALKFTSQIETLGRKVVSILRQNGPFVVLHLRYEMDMLAFSGCSHGCNNEEVEHLTRMRYAYPWWKEKVIDSDFKRREGLCPLTPEETAQVLTALGINRDVQIYIAAGEIYGGARRMASLAAAFPNLVRKENLLDPSDLRYFQNHSSQMAALDYLVSLQSDIFVPTYDGNMAKVVEGHRRYLGFKKTISLNRKLLVDLIDRYHGGLLSWDDFSSMVKESHADRMGSAKQRLVIPDRPKEEDYFYANPHECFQPLVEQYTLE

SEQ ID NO: 30 - DNA, 39 bp, synthetic construct
  source 1..39: mol_type = other DNA
taagaaggagatataccatgaccagcagtcagctgaaag

SEQ ID NO: 31 - DNA, 41 bp, synthetic construct
  source 1..41: mol_type = other DNA
ctgttcgacttaagcattacttattcagcagcggacccaca

SEQ ID NO: 32 - DNA, 64 bp, synthetic construct
  source 1..64: mol_type = other DNA
taagaaggagatataccatggatgatctctcttctctaaaactgggtgttaaattttttcaagc

SEQ ID NO: 33 - DNA, 48 bp, synthetic construct
  source 1..48: mol_type = other DNA
ctgttcgacttaagcattactttcccttaacaaacggcggacttccac

SEQ ID NO: 34 - DNA, 46 bp, synthetic construct
  source 1..46: mol_type = other DNA
ttaagaaggagatataccatggctaccaatattgagcagcagcagc

SEQ ID NO: 35 - DNA, 46 bp, synthetic construct; identical to SEQ ID NO: 34
  source 1..46: mol_type = other DNA

SEQ ID NO: 36 - DNA, 60 bp, synthetic construct
  source 1..60: mol_type = other DNA
ttaagaaggagatataccatggattcacaatttcagcagcttcactttgttatgataccc

SEQ ID NO: 37 - DNA, 61 bp, synthetic construct
  source 1..61: mol_type = other DNA
ctgttcgacttaagcattagcttaatgctaacctatcctttacttgttcaatgatgtcttg

SEQ ID NO: 38 - DNA, 47 bp, synthetic construct
  source 1..47: mol_type = other DNA
taagaaggagatataccatgggttcagaatctcaagtgcagcttcac

SEQ ID NO: 39 - DNA, 54 bp, synthetic construct
  source 1..54: mol_type = other DNA
gttcgacttaagcatcacttcctttctttcaaaatgtcttggattagtaacgtc

SEQ ID NO: 40 - DNA, 54 bp, synthetic construct
  source 1..54: mol_type = other DNA
taagaaggagatataccatggccaacttagctcaaaaccttcattttgtcttgc

SEQ ID NO: 41 - DNA, 60 bp, synthetic construct
  source 1..60: mol_type = other DNA
ttctgttcgacttaagcatcatttacttgtcccatgggccataatttcttgaattaggtg

SEQ ID NO: 42 - DNA, 36 bp, synthetic construct
  source 1..36: mol_type = other DNA
taagaaggagatataccatgtcccgaatcagcggtc

SEQ ID NO: 43 - DNA, 46 bp, synthetic construct
  source 1..46: mol_type = other DNA
tgttcgacttaagcatcaaacattttcttgattgttcaatttccgg

SEQ ID NO: 44 - DNA, 48 bp, synthetic construct
  source 1..48: mol_type = other DNA
taagaaggagatataccatgggaactattgcaaacggagaaattgccc

SEQ ID NO: 45 - DNA, 57 bp, synthetic construct
  source 1..57: mol_type = other DNA
gttcgacttaagcatcaacaagtggtaattgatctaatttctgaaatgaattcgtcg

SEQ ID NO: 46 - DNA, 47 bp, synthetic construct
  source 1..47: mol_type = other DNA
aagaaggagatatacaatggattctcaatttcaacaattgcattttg

SEQ ID NO: 47 - DNA, 62 bp, synthetic construct
  source 1..62: mol_type = other DNA
cagcagcctaggttaatttaagacaaagctaatctatctttaacttgttcaattatatcttg

SEQ ID NO: 48 - DNA, 63 bp, synthetic construct
  source 1..63: mol_type = other DNA
gttgaaattgagaatccatggtatatctccttcttaaagttaaacaaaattatttctagaggg

SEQ ID NO: 49 - DNA, 43 bp, synthetic construct
  source 1..43: mol_type = other DNA
gatagattagctttgtcttaagaattcgagctccgtcgacaag

SEQ ID NO: 50 - DNA, 44 bp, synthetic construct
  source 1..44: mol_type = other DNA
gtatatctccttcttaaagttaaacaaaattatttctagagggg

SEQ ID NO: 51 - DNA, 31 bp, synthetic construct
  source 1..31: mol_type = other DNA
tgcttaagtcgaacagaaagtaatcgtattg

SEQ ID NO: 52 - DNA, 50 bp, synthetic construct
  source 1..50: mol_type = other DNA
taataaggagatataccatggaggttagatccgagagtgtacagttgagg

SEQ ID NO: 53 - DNA, 57 bp, synthetic construct
  source 1..57: mol_type = other DNA
gattactttctgttcgatcatctcgaagtttctgttgaattgacggtacctaatacg

SEQ ID NO: 54 - DNA, 54 bp, synthetic construct
  source 1..54: mol_type = other DNA
taataaggagatataccatgtgtgaattagatgagaacagggaagagagggaga

SEQ ID NO: 55 - DNA, 59 bp, synthetic construct
  source 1..59: mol_type = other DNA
gattactttctgttcgatcatgtaattcttaatggttcaaccaagggttgcaaacactc

SEQ ID NO: 56 - DNA, 60 bp, synthetic construct
  source 1..60: mol_type = other DNA
ataaggagatataccatggagtttagatctgagaatgcacagaataggtgtgataaactg

SEQ ID NO: 57 - DNA, 49 bp, synthetic construct
  source 1..49: mol_type = other DNA
cgattactttctgttcgattaccggaacttctcagttaaattgcccggg

SEQ ID NO: 58 - DNA, 49 bp, synthetic construct
  source 1..49: mol_type = other DNA
taataaggagatataccatggggtggaagacattaggaggtgagagtag

SEQ ID NO: 59 - DNA, 60 bp, synthetic construct
  source 1..60: mol_type = other DNA
tactttctgttcgatcagatgatgcttagaagttcttcagatgaaacttctgaaaatcgc

SEQ ID NO: 60 - DNA, 46 bp, synthetic construct
  source 1..46: mol_type = other DNA
aataaggagatataccatgaaaagtgtgagagtggaggggagaggg

SEQ ID NO: 61 - DNA, 57 bp, synthetic construct
  source 1..57: mol_type = other DNA
gattactttctgttcgatcattcaagtgtatattgttcaaccaagggctgaaaacac

SEQ ID NO: 62 - DNA, 43 bp, synthetic construct
  source 1..43: mol_type = other DNA
tttaactttaataaggagatataccatggcgacccataccccg

SEQ ID NO: 63 - DNA, 39 bp, synthetic construct
  source 1..39: mol_type = other DNA
catggtatatctccttttactcgagcgctttcacttcgg

SEQ ID NO: 64 - DNA, 59 bp, synthetic construct
  source 1..59: mol_type = other DNA
agttgctggcgctgattggcatcgttttagagctagaaatagcaagttaaaataaggct

SEQ ID NO: 65 - DNA, 51 bp, synthetic construct
  source 1..51: mol_type = other DNA
aaacgatgccaatcagcgccagcactagtattatacctaggactgagctag

SEQ ID NO: 66 - DNA, 24 bp, synthetic construct
  source 1..24: mol_type = other DNA
cctcgtgtcaggggatccattttc

SEQ ID NO: 67 - DNA, 50 bp, synthetic construct
  source 1..50: mol_type = other DNA
tgatccggcaaacaaaccaccgctggtagccacggcgcggttttcagtgc

SEQ ID NO: 68 - DNA, 54 bp, synthetic construct
  source 1..54: mol_type = other DNA
gctaccagcggtggtttgtttgccggatcattgagcaggaatatcgtgatcagg

SEQ ID NO: 69 - DNA, 23 bp, synthetic construct
  source 1..23: mol_type = other DNA
tttacccaaaaacatttcgggcg